(19) United States
(12) Patent Application Publication
(10) Pub. No.: US 2024/0379225 A1
(43) Pub. Date: Nov. 14, 2024

(54) DETERMINING A STATE OF A MEDICAL CONDITION BASED ON A MICROBIOME SAMPLE

(71) Applicant: Bar-Ilan University, Ramat-Gan (IL)

(72) Inventor: Yoram LOUZOUN, Elkana (IL)

(73) Assignee: Bar-Ilan University, Ramat-Gan (IL)

(21) Appl. No.: 18/195,980

(22) Filed: May 11, 2023

(51) Int. Cl. G16H 50/20; G16B 20/00; G16B 45/00

(52) U.S. Cl. CPC G16H 50/20; G16B 20/00; G16B 45/00

(57) ABSTRACT

There is provided a computer implemented method of determining a state of a medical condition of a subject, comprising: computing a tree that includes representations computed from genetic sequences of a plurality of microbes within a microbiome sample of a subject, wherein the tree includes the representations of the plurality of microbes arranged according to a taxonomy hierarchy, mapping the tree to a data structure that defines the taxonomy hierarchy and defines positions between neighboring representations of the plurality of microbes, feeding the data structure into a machine learning model that processes neighboring representations according to position, and obtaining the state of the medical condition of the subject as an outcome of the machine learning model.

### BACKGROUND

The present invention, in some embodiments thereof, relates to machine learning and, more specifically, but not exclusively, to a machine learning approaches for analysis of microbiomes.

The microbiome is often analyzed using 16S rRNA gene sequencing. The 16S gene sequences may be represented as features counts, which are fed into a machine learning model.

### SUMMARY

According to a first aspect, a computer implemented method of determining a state of a medical condition of a subject, comprises: computing a tree that includes representations computed from genetic sequences of a plurality of microbes within a microbiome sample of a subject, wherein the tree includes the representations of the plurality of microbes arranged according to a taxonomy hierarchy, mapping the tree to a data structure that defines the taxonomy hierarchy and defines positions between neighboring representations of the plurality of microbes, feeding the data structure into a machine learning model that processes neighboring representations according to position, and obtaining the state of the medical condition of the subject as an outcome of the machine learning model.

According to a second aspect, a computer implemented method of training a machine learning model for determining a state of a medical condition of a subject, comprises: obtaining a plurality of sample microbiome samples from a plurality of subjects, computing a tree that includes representations computed from genetic sequences of a plurality of microbes within the plurality of sample microbiome sample, wherein the tree includes the representations of the plurality of microbes arranged according to a taxonomy hierarchy, mapping the tree to a data structure that defines the taxonomy hierarchy and defines positions between neighboring representations of the plurality of microbes, creating a training dataset of a plurality of records, wherein a record comprises the data structure and a ground truth indication of the state of a medical condition of a subject from the plurality of subjects, and training the machine learning model on the training dataset, wherein the machine learning model processes neighboring representations according to position, wherein the machine learning model generates the state of the medical condition of the subject in response to an input of a data structure computed from a microbiome sample obtained from the subject.

According to a third aspect, a system for determining a state of a medical condition of a subject, comprises: computing a tree that includes representations computed from genetic sequences of a plurality of microbes within a microbiome sample of a subject, wherein the tree includes the representations of the plurality of microbes arranged according to a taxonomy hierarchy, mapping the tree to a data structure that defines the taxonomy hierarchy and defines positions between neighboring representations of the plurality of microbes, feeding the data structure into a machine learning model that processes neighboring representations according to position, and obtaining the state of the medical condition of the subject as an outcome of the machine learning model, wherein the machine learning model is trained by: obtaining a plurality of sample microbiome samples from a plurality of subjects, computing the tree that includes representations computed from genetic sequences of the plurality of sample microbiome samples, mapping the tree to the data structure, creating a training dataset of a plurality of records, wherein a record comprises the data structure a and a ground truth indication of the state of a medical condition of a subject from the plurality of subjects, and training the machine learning model on the training dataset.

In a further implementation form of the first, second, and third aspects, further comprising ordering elements of the data structure each denoting a type of microbe according to at least one similarity feature between different types of microbes at a same taxonomy level of the taxonomy hierarchy while preserving the structure of the tree.

In a further implementation form of the first, second, and third aspects, the ordering is done per taxonomic category level, for positioning the representations of microbes that are more similar closer together and representation of microbes that are less similar are positioned further apart while preserving the structure of the tree.

In a further implementation form of the first, second, and third aspects, the ordering is computed recursively per taxonomic category level.

In a further implementation form of the first, second, and third aspects, the ordering is done for positioning taxa with similar frequencies relatively closer together and taxa with less similar frequencies further apart.

In a further implementation form of the first, second, and third aspects, the at least one similarity feature includes similarity of frequency of microbes.

In a further implementation form of the first, second, and third aspects, the at least one similarity feature is computed based on Euclidean distances.

In a further implementation form of the first, second, and third aspects, the at least one similarity feature is computed by building a dendrogram based on the Euclidean distances computed for a hierarchical clustering of the representations of the microbes according to frequency.

In a further implementation form of the first, second, and third aspects, the machine learning model is trained by: obtaining a plurality of sample microbiome samples from a plurality of subjects, computing the tree that includes representations computed from genetic sequences of the plurality of sample microbiome samples, mapping the tree to the data structure, creating a training dataset of a plurality of records, wherein a record comprises the data structure and a ground truth indication of the state of the medical condition of a subject from the plurality of subjects, and training the machine learning model on the training dataset.

In a further implementation form of the first, second, and third aspects, the tree is created by including each observed taxa of microbes of the microbiome sample in a leaf at a respective taxonomic level of the taxonomy hierarchy, and adding to each leaf a log-normalized frequency of the observed taxa of microbes, and each internal node includes an average of direct descendants of the internal node located at lower levels.

In a further implementation form of the first, second, and third aspects, the data structure is implemented as a graph that includes values of the tree and an adjacent matrix, that are fed into a layer of a graph convolutional neural network implementation of the machine learning model, wherein an identity matrix is added with a learned coefficient, and output of the layer is fed into a fully connected layer that generates the outcome.

In a further implementation form of the first, second, and third aspects, the data structure is represented as an image created by projecting the tree to a two dimensional matrix with a plurality of rows matching a number of different taxonomy category levels of the taxonomy hierarchy represented by the tree, and a column for each leaf, wherein the machine learning model is implemented as a convolutional neural network.

In a further implementation form of the first, second, and third aspects, at each respective level of the different taxonomy category levels, a value of the two dimensional matrix at the respective level is set to the value of leaves below the level or a value at a higher level is set to have an average of values of a level below, and below a leaf values are set to zero, and wherein a level below includes a plurality of different values, a plurality of positions of the two dimensional matrix of the current level are set to an average of the plurality of different values of the level below.

In a further implementation form of the first, second, and third aspects, further comprising applying an explainable AI platform to the machine learning model for obtaining an estimate of portions of the data structure used by the machine learning model for obtaining the outcome, and projecting the portions of the data structure to the tree for obtaining an indication of which microorganisms of the microbiome most contributed to the outcome of the machine learning model.

In a further implementation form of the first, second, and third aspects, further comprising iterating the applying the explainable AI platform for each of a plurality of data structures of sequentially obtained microbiome samples, for creating a plurality of heatmaps each at a different color channel, and combining the plurality of heatmaps into a single multi-channel image, and projecting the multi-channel image on the tree.

In a further implementation form of the first, second, and third aspects, a plurality of trees are computed for a plurality of microbiome samples obtained at spaced apart time intervals, wherein the plurality of trees are mapped to a plurality of image representations of the data structures corresponding to the spaced apart time intervals, the plurality of images are combined into a 3D image depicting temporal data, and the 3D image is fed into a 3D-CNN implementation of the machine learning model.

In a further implementation form of the first, second, and third aspects, further comprising pre-processing the genetic sequences by clustering the genetic sequences to create Amplicon Sequence Variants (ASVs), and creating a vector of the ASVs, wherein each entry of the vector represents a microbe at a certain taxonomy level of the taxonomy hierarchy and comprises the representation, wherein the tree is created from the vector by placing respective values of the vector at corresponding taxonomic category levels of the taxonomy hierarchy of the tree.

In a further implementation form of the first, second, and third aspects, further comprising computing a log-normalization of the ASV frequencies of the vector to obtain a log-normalized vector of ASV frequencies, wherein the tree includes the values of the log-normalized vector.

In a further implementation form of the first, second, and third aspects, further comprising recursively ordering the log-normalized ASV frequencies according to at least one similarity feature computed based on Euclidean distances between log-normalized ASV frequencies.

In a further implementation form of the first, second, and third aspects, the medical state of the subject is an indication of a complication that arises during pregnancy that is linked to lifelong health risks, including at least one of: gestational diabetes, preeclampsia, preterm birth, and postpartum depression.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a block diagram of a system for computing a medical state of a subject based on a cladogram created from genetic sequences of a sample of a microbiome and/or training an ML model for computing the medical state of the subject based on the cladogram, in accordance with some embodiments of the present invention;

FIG. 2 is a flowchart of an exemplary method of computing a medical state of a subject based on a cladogram created from genetic sequences of a sample of a microbiome, in accordance with some embodiments of the present invention;

FIG. 3 is a flowchart of an exemplary method of training an ML model for computing a medical state of a subject based on a cladogram created from genetic sequences of a sample of a microbiome, in accordance with some embodiments of the present invention;

FIG. 4 is a table presenting comparison between existing methods' ability to address technical problems described herein, in accordance with some embodiments of the present invention;

FIG. 5 is a pseudocode of an exemplary approach for populating a mean cladogram, in accordance with some embodiments of the present invention;

FIG. 6 is a pseudocode of an exemplary approach for mapping the cladogram to a matrix, in accordance with some embodiments of the present invention;

FIG. 7 is a pseudocode of an exemplary reordering approach, in accordance with some embodiments of the present invention;

FIG. 8 is a table of examples of additional features that may be fed in combination with the image into the ML model, in accordance with some embodiments of the present invention.

FIG. 9 is a schematic of an exemplary dataflow of gMic+v, in accordance with some embodiments of the present invention;

FIG. 10 is a schematic of an exemplary dataflow of iMic, in accordance with some embodiments of the present invention;

FIG. 11 is a schematic of an exemplary dataflow of a 3D implementation of iMic, in accordance with some embodiments of the present invention;

FIG. 12 includes Grad-Cam images, in accordance with some embodiments of the present invention;

FIG. 13 includes cladogram projections, in accordance with some embodiments of the present invention;

FIG. 14 includes heatmaps created from three images, in accordance with some embodiments of the present invention;

FIG. 15 includes Grad-Cam projections of heatmaps described with reference to FIG. 14 on the cladogram, in accordance with some embodiments of the present invention;

FIG. 16 is a table of datasets used in the experiment, in accordance with some embodiments of the present invention;

FIG. 17 is a table of the sequential datasets used in the experiment, in accordance with some embodiments of the present invention;

FIG. 18 is a graph presenting a comparison between model performance performed as part of the Experiment described herein, in accordance with some embodiments of the present invention;

FIG. 19 is a table of 10 CV mean performances based on experimental results, in accordance with some embodiments of the present invention;

FIG. 20 includes graphs indicating that iMic copes with the ML challenges above better than other methods based on experimental results, in accordance with some embodiments of the present invention;

FIG. 21 includes graphs indicating importance of ordering taxa based on experimental results, in accordance with some embodiments of the present invention;

FIG. 22 includes graphs depicting interpretation based on experimental results, in accordance with some embodiments of the present invention; and

FIG. 23 includes graphs comparing performance of 3D learning vs PhyLoSTM based on experimental results, in accordance with some embodiments of the present invention.

### DETAILED DESCRIPTION

The present invention, in some embodiments thereof, relates to machine learning and, more specifically, but not exclusively, to a machine learning approaches for analysis of microbiomes.

As used herein, the term iMic refers to embodiments based on an image, and the term gMic refers to embodiments based on a graph. Exemplary processes of iMic and/or gMic are described herein.

As used herein, the term tree may refer, for example, to a cladogram and/or taxonomy tree. The terms tree and cladogram and taxonomy tree may be used interchangeably.

As used herein, the term data structure may refer, for example, to an image and/or two dimensional matrix and/or graph. The terms data structure and image may be used interchangeably, where features of the data structure apply to an image.

The terms data structure and image may be used interchangeably, for example, where features of the data structure apply to an image, the term image may be used. In another example, where features of the data structure apply to a graph, the term graph may be used. The terms data structure, image, and graph may be used interchangeable where similar processing is done for images and graphs.

An aspect of some embodiments of the present invention relates to systems, methods, computing devices, and/or code instructions (stored on a data storage device and executable by one or more processors) for determining a state of a medical condition of a subject. A processor accesses genetic sequences of microbes within a microbiome sample of a subject. The microbiome sample may be obtained, for example, from stool, a vaginal sample, from spit, and the like. The genetic sequences may be pre-processed to compute representations of the microbes, for example, a vector of log-normalized amplicon sequence variants (ASVs). A tree (e.g., cladogram, taxonomy tree) that includes the representations of the microbes arranged according to a taxonomy hierarchy is created. The tree is mapped to a data structure that defines the taxonomy hierarchy and defines positions between neighboring representations of the microbes, for example, an image and/or a graph. It is noted that the tree does not define positions between neighboring representation of the microbes, since different positions do not impact the structure of the tree, while different positions impact the structure of the image and/or graph. The data structure, optionally the graph and/or image, is fed into a machine learning model that processes neighboring representations according to position. For example, a convolutional neural network (CNN) is fed images. The CNN applies filters which are impacted by the location of pixels of the image that correspond to different types of microbes. The state of the medical condition of the subject is obtained as an outcome of the machine learning model.

Optionally, elements of the data structure each denoting a type of microbe (e.g., pixels of the image) are ordered according to one or more similarity features between different types of microbes while preserving the structure of the tree. The ordering may be done per taxonomic category level, for positioning the representations of microbes that are more similar closer together and representation of microbes that are less similar are positioned further apart while preserving the structure of the tree. The ordering may be computed recursively per taxonomic category level. The similarity feature(s) may be computed by building a dendrogram based on Euclidean distances computed for a hierarchical clustering of the representations of the microbes.

Inventors discovered that feeding the data structure, optionally ordered, into the ML model improves performance of the ML model, for example, improving accuracy of a correct prediction of the state of the medical condition. Inventors hypothesize that ordering the pixels of the image according to similarity, followed by processing by a CNN, enables the ML model to analyze groups of similar microbes which are located in proximity to one another, which increases the performance of the ML model.

An aspect of some embodiments of the present invention relates to systems, methods, computing devices, and/or code instructions (stored on a data storage device and executable by one or more processors) for training the ML model that generates the outcome of the state of the medical condition of a subject in response to an input of the data structure (e.g., image and/or pixel), optionally ordered. Multiple sample microbiome samples are obtained from multiple subjects. The tree (e.g., cladogram, taxonomy tree) that includes representations computed from genetic sequences of the multiple sample microbiome samples is created. The tree is mapped to the data structure (e.g., image and/or graph). A training dataset of multiple records is created. A record includes the data structure and a ground truth indication of the state of the medical condition of a subject from the multiple subjects is created. Multiple records may be created for the multiple microbiome samples and/or multiple subjects. The machine learning model (e.g., CNN, GNN) is trained on the training dataset.

At least some embodiments of the systems, methods, computing devices, and/or code instructions (i.e., stored on a data storage device and executable by one or more processors) described herein address the technical problem of improving diagnosis of a medical condition of a subject based on an analysis of genetic sequences of microbiomes in a biological sample obtained from the subject. At least some embodiments of the systems, methods, computing devices, and/or code instructions described herein improve the medical field of diagnosis a medical condition of a subject. At least some embodiments of the systems, methods, computing devices, and/or code instructions described herein improve upon prior approaches of analyzing genetic sequences of microbiomes in a biological sample obtained from the subject.

At least some embodiments of the systems, methods, computing devices, and/or code instructions described herein address the technical problem of increasing performance of a ML model that is fed a data structure based on genetic sequences of microbiomes in a biological sample obtained from the subject, and generates an outcome, for example, diagnosis of a medical condition. At least some embodiments of the systems, methods, computing devices, and/or code instructions described herein improve the technical field of machine learning. At least some embodiments of the systems, methods, computing devices, and/or code instructions described herein improve upon prior approaches of using ML models fed a data structure based on genetic sequences of microbiomes in a biological sample obtained from the subject, and generates an outcome.

The human gut microbial composition is associated with many aspects of human health (e.g., [1, 2, 3, 4, 5, 6]). This microbial composition is often determined through sequencing of the 16S rRNA gene [7, 8] or shotgun metagenomics [9, 10, 11]. In some standard approaches, the sequences are then clustered to produce Amplicon Sequence Variants (ASVs), which in turn are associated with taxa [12]. This association is often not species or strain specific, but rather resolved to broader taxonomic levels (Phylum, Class, Order, Family, and Genus) [13, 14]. The sequence-based microbial compositions of a sample have often been proposed as biomarkers for diseases [15, 16, 17]. Such associations can be translated to ML (machine learning)-based predictions, relating the microbial composition to different conditions [18, 19, 20, 21]. However, multiple technical problems limit the accuracy of ML in microbiome studies. For example, first, the usage of ASVs as predictors of a condition requires the combination of information at different taxonomic levels. Also, in typical microbiome experiments, there are tens to hundreds of samples vs thousands of different ASVs. Finally, the ASVs are sparse, while a typical experiment can contain thousands of different ASVs. Most ASVs are absent from the vast majority of samples.

In an attempt to overcome these technical problems, prior data aggregation methods have been proposed, where the hierarchical structure of the cladogram (taxonomic tree) can be used to combine different ASVs [14, 22]. For example, a class of phylogenetic-based feature weighting algorithms was proposed to group relevant taxa into clades, and the high weights clade groups were used to classify samples with a random forest (RF) algorithm [23]. An alternative method is a taxonomy-based smoothness penalty to smooth the coefficients of the microbial taxa with respect to the cladogram in both linear and logistic regression models [24]. However, these simple models do not resolve the sparsity of the data and make limited use of the taxonomy.

Deep neural networks (DNNs) were proposed to identify more complex relationships among microbial taxa. Typically, the relative ASVs vectors are the input of a multi-layer perceptron neural network (MLPNN) or recursive neural network (RNN) [25]. However, given the typical distribution of microbial frequencies, these methods end up using mainly the prevalent and abundant microbes and ignore the wealth of information available in rare taxa.

At least some embodiments described herein address the aforementioned technical problem(s), and/or improve upon the aforementioned technical field(s), and/or improve upon the aforementioned prior approaches(s), by using the cladogram to translate the microbiome to graphs and/or images. In the images, an iterative ordering approaches is implemented to ensure that taxa with similar frequencies among samples are neighbors in the image. ML model(s) (e.g., Convolutional Neural Networks (CNNs) [26] and/or Graph Convolutional Networks (GCNs) [27]) are applied to the classification of such samples. Both CNN and GCN use convolution over neighboring nodes to obtain an aggregated measure of values over an entire region of the input. The difference between the two is that CNNs aggregate over neighboring pixels in an image, while GCNs aggregate over neighbors in a graph. CNNs have been successfully applied to diversified areas such as face recognition [28], optical character recognition [29] and medical diagnosis [30].

Several previous models combined microbiome abundances using CNNs [31, 32, 33, 34], using approaches that are different than embodiments described herein. For example, PopPhy-CNN constructs a phylogenetic tree to preserve the relationship among the microbial taxa in the profiles. The tree is then populated with the relative abundances of microbial taxa in each individual profile and represented in a two-dimensional matrix as a natural projection of the phylogenetic tree in R2. Taxon-NN [32] stratifies the input ASVs into clusters based on their phylum information and then performs an ensemble of 1D-CNNs over the stratified clusters containing ASVs from the same phylum. As such, more detailed information on the species level representation is lost. Deep ensemble learning over the microbial phylogenetic tree (DeepEn-Phy) [34] is probably the most extreme usage of the cladogram, since the neural network is trained on the cladogram itself. As such, it is fully structured to learn the details of the cladogram. TopoPhy-CNN [33] also utilizes the phylogenetic tree topological information for its predictions similar to PopPhy. However, TopoPhy-CNN assigns a different weight to different nodes in the tree (hubs get higher weights, or weights according to the distance in the tree). Finally, CoDaCoRe [35] identifies sparse, interpretable, and predictive log-ratio biomarkers. The algorithm exploits a continuous relaxation to approximate the underlying combinatorial optimization problem. This relaxation can then be optimized by using gradient descent.

Graph ML and specifically GCN-based graph classification tasks have rarely been used in the context of microbiome analysis [36], but may also be considered for microbiome-based classification. Graph classification methods which may be implemented in one or more embodiments described herein include, for example, (see also [37, 38, 39]), DIFFPOOL, a differentiable graph pooling module that can generate hierarchical representations of graphs and use 79 this hierarchy of vertex groups to classify graphs [40]. StructPool [41] considers graph pooling as a vertex clustering problem. EigenGCN [42] proposes a pooling operator. EigenPooling is based on the graph Fourier transform, which can utilize the vertex features and local structures during the pooling process, and QGCN [43] using a quadratic formalism in the last layer. At least some embodiments described herein use GCNs for microbiome classification, which is different than prior approaches where GCNs have never been used for microbiome classification.

At least some embodiments describes herein directly integrate the cladogram and the measured microbial frequencies into either a graph or an image to produce gMic and iMic (graph Microbiome and image Microbiome). In the Experiment section, Inventors show that the relation between the taxa present in a sample is often as informative as the frequency of each microbe (gMic) and that this relation can be used to significantly improve the quality of ML-based prediction in microbiome-based biomarker development (micmarkers) over current state-of-the-art methods (iMic). iMic provides technical solutions to the technical problems discussed herein (e.g., different levels of representation, sparsity, and a small number of samples). iMic and gMic are accessible at https://github(dot)com/oshritshtossel/iMic. iMic is also available as a python package via PyPI, under the name MIPMLP.micro2matrix and MIPMPLP. CNN2, https://pypi?(dot)org/project/MIPMLP/.

Three main components can be proposed to solve the technical problems described herein, in particular ((1) non-uniform representation; (2) a small number of samples compared with the dimension of each sample; and (3) sparsity of the data, with the majority of taxa present in a small subset of samples).

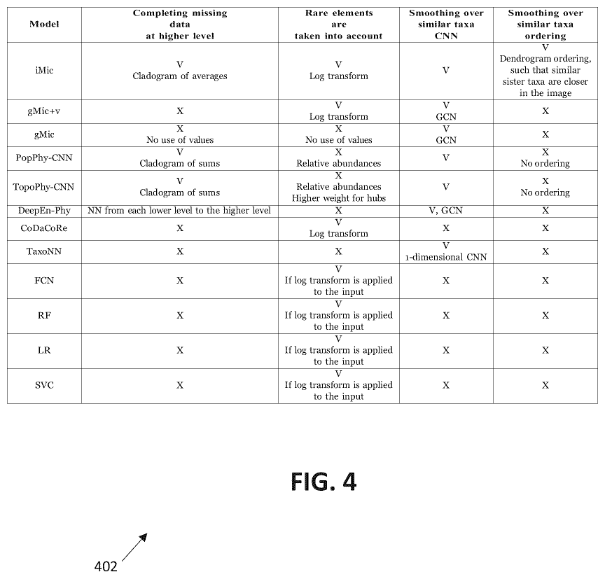

Referring now back to FIG. 4, table 199 presents a comparison between existing methods' ability to address technical problems described herein. Table 199 indicates that prior method address all of the technical problems described herein, in comparison to embodiments based on ML models described herein which do address all or major technical problems described herein, for example:

(1) Completion of missing data at a higher level, such that even information that is sparse at a fine taxonomy level is not sparse at a broad level. Most of the value-based methods (fully connected neural network (FCN), RF, logistic regression (LR), and Support Vector Machine classifier (SVC)) use only a certain taxonomy level (usually genus or species) and do not cope with missing data at this level. More sophisticated methods (CodaCore and TaxoNN) do not complete missing data. iMic (average), PopPhy-CNN and TopoPhy-CNN (sum) use the phylogenetic tree structure to fill in missing data at a broad taxonomy level. DeepEn-Phy completes the missing taxa by building neural networks between the finer and coarse levels in the cladogram.

(2) The incorporation of rare taxa. The log transform ensures that even rare elements are taken into account. The relative abundance of the microbiome is practically not affected by rare taxa which can be important [44, 45]. Log-transform can be applied to the input of most of the models, as can be seen in our implementation of all the basic models. However, none of the structure-based methods except for iMic and gMic works with the logged values.

(3) Smoothing over similar taxa to ensure that even if some values are missing, they can be completed by their neighbors. This is obtained in iMic by the combination of the CNN and the ordering, reducing the sensitivity to each taxon. Either ordering or CNN by themselves is not enough to handle the sparsity of the samples. Multiple CNN-based methods have been proposed. However, none of the methods besides iMic reorder the taxa such that more similar taxa will be closer. Note that other solutions that would do similar smoothing would probably get the same effect.

As discussed, microbiome-based ML is hindered by multiple technical challenges, including for example, several representation levels, high sparsity, and high dimensional input vs a small number of samples. At least some embodiments described herein based on iMic solve these technical challenges, by simultaneously using (e.g., all) known taxonomic levels. Moreover, iMic resolves sparsity by ensuring ASVs with similar taxonomy are nearby and averaged at finer taxonomic levels. As such, even if each sample has different ASVs, there is still common information at finer taxonomic levels. For example:
High sparsity. The microbiome data is extremely sparse with most of the taxa absent from most of the samples. iMic CNN averages over neighboring taxa. As such, even in the absence of some taxa, it can still infer their expected value from their neighbors.
High dimensional input vs a small number of samples. By using CNNs with strides on the image microbial input, iMic reduces the model's number of parameters in comparison to FCNs.
Several representation levels. iMic uses all taxonomic levels by adding the structure of the cladogram and translating it into an image. iMic further finds the best representation as an image by reordering the columns of the rows using for example, dendrogram clustering while maintaining the taxonomy structure.

As discussed herein, the application of ML to microbial frequencies, for example represented by 16S rRNA and/or shotgun metagenomics ASV counts at a specific taxonomic level is affected for example, by 3 types of information loss—ignoring the taxonomic relationships between taxa, ignoring sparse taxa present in only a few samples, and ignoring rare taxa (taxa with low frequencies) in general.

As described herein, the cladogram used in at least some embodiments is highly informative through a graph-based approach termed gMic. Even completely ignoring the frequency of the different taxa, and optionally only using their absence or presence, may lead to highly accurate predictions on multiple ML tasks, typically as good or even better than the current state-of-the-art.

As described in the Experiment section, embodiments based on iMic produce higher precision predictions than current state-of-the-art microbiome-based ML on a wide variety of ML tasks. iMic is less sensitive to the limitations above. Specifically, iMic is less sensitive to the rarefaction of the ASV in each sample. Removing random taxa from samples had the least effect on iMic's accuracy in comparison to other methods. Similarly, iMic is most robust to the removal of full samples. Finally, iMic explicitly incorporates the cladogram. Removing the cladogram information reduces the classification accuracy. A typical window of 3 snapshots may be enough to extract the information from dynamic microbiome samples.

An potential advantage of iMic is the production of explainable models. Moreover, treating the microbiome as images opens the door to many vision-based ML tools, such as: transfer learning from pre-trained models on images, self-supervised learning, and data augmentation. Combining iMic with an explainable AI methodology may highlight microbial taxa associated as a group with different phenotypes.

The development of microbiome-based biomarkers (micmarkers) is one of the most promising routes for easy and large-scale detection and prediction. However, while many microbiome based prediction algorithms have been developed, they suffer from multiple limitations, which are mainly the result of the sparsity and the skewed distribution of taxa in each host. Embodiments described herein based on iMic and/or gMic are for translation of microbiome samples from a list of single taxa to a more holistic view of the full microbiome.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

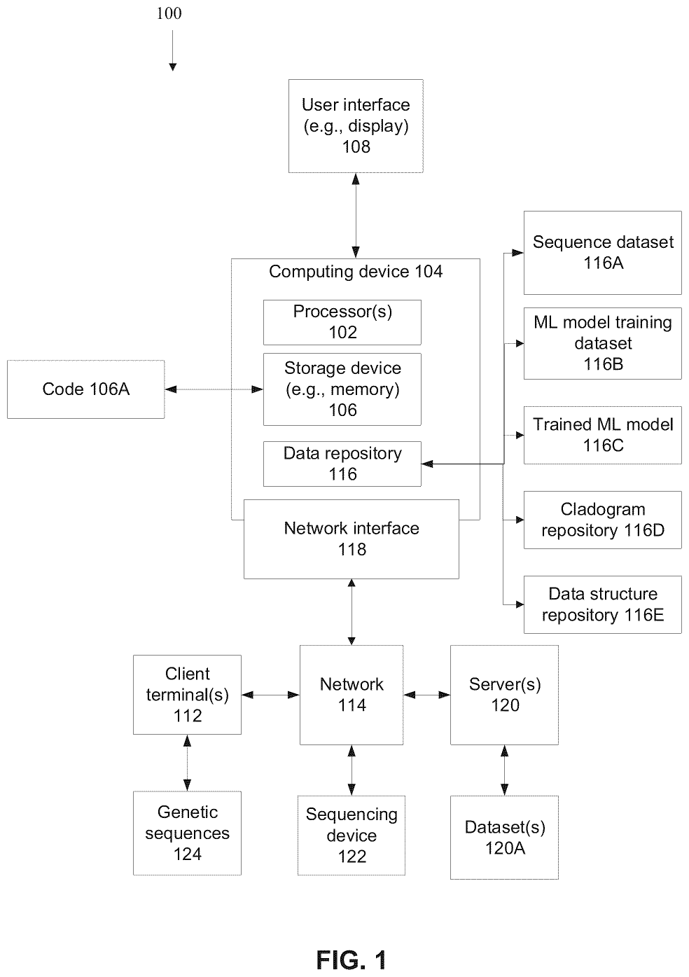

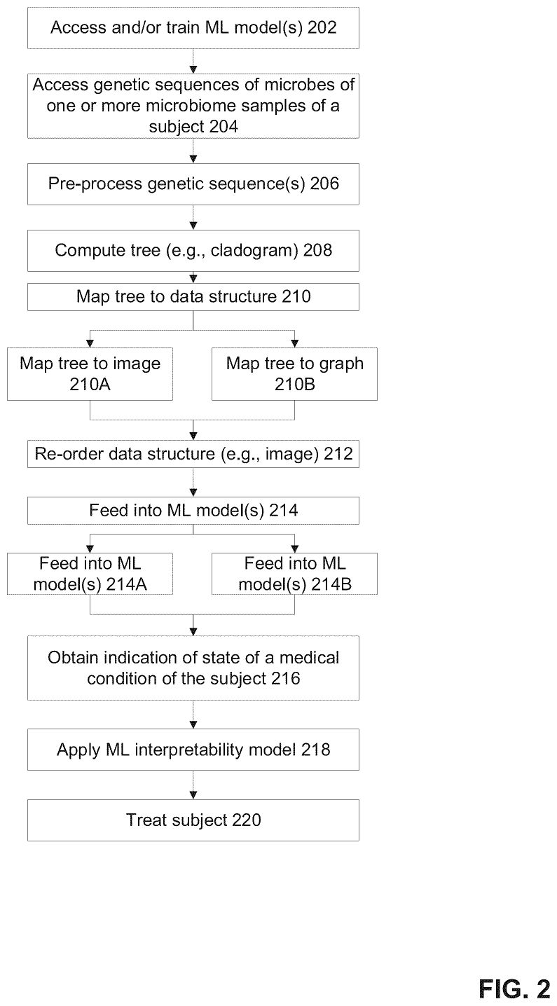

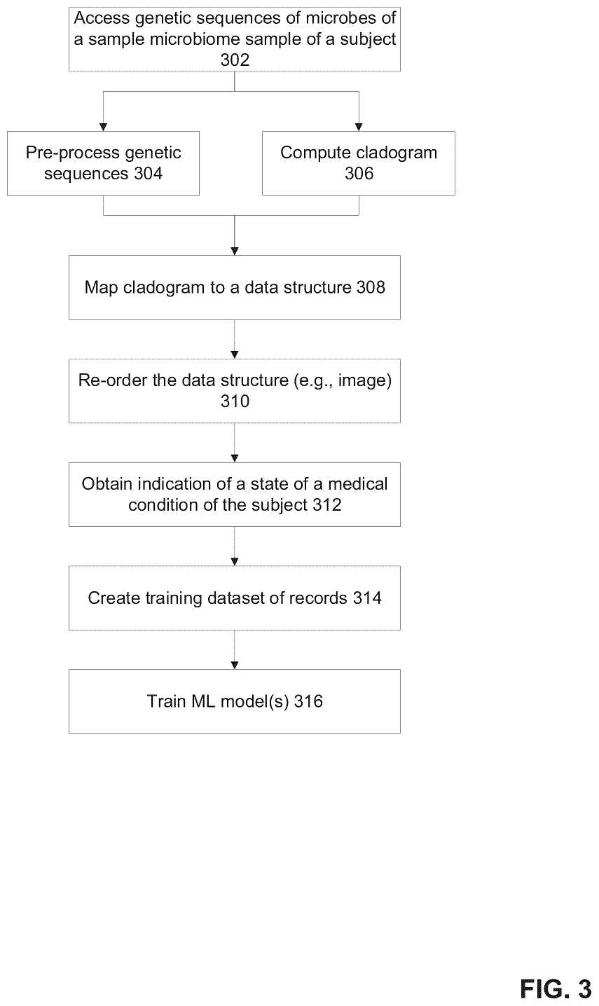

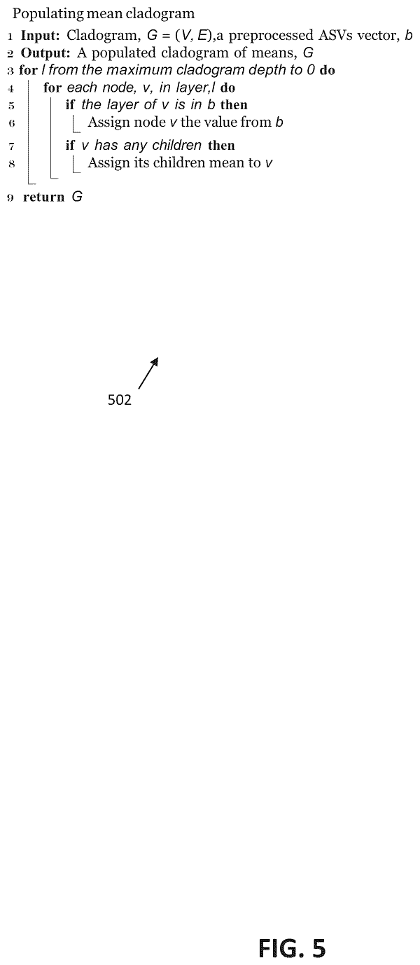

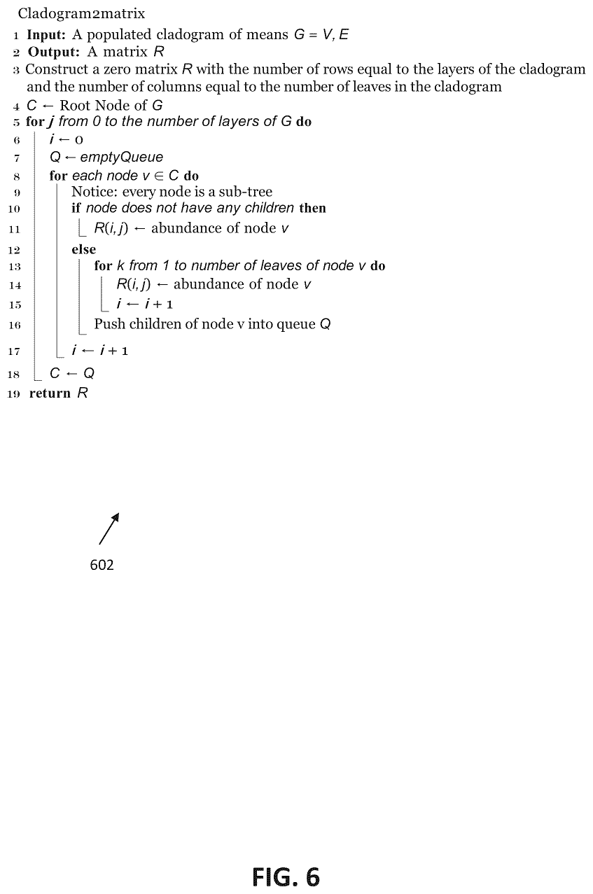

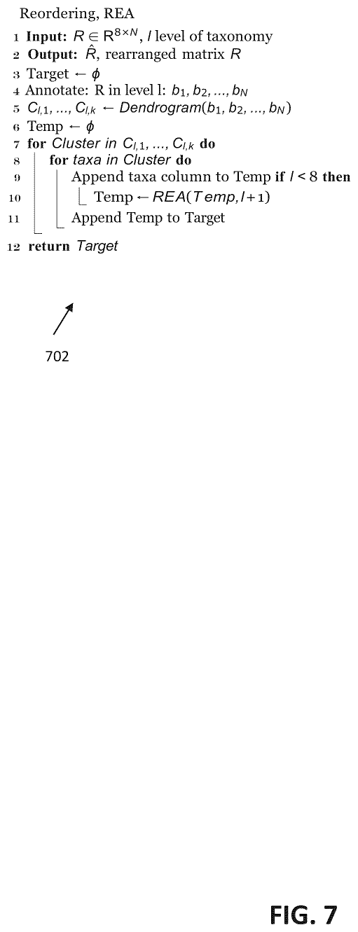

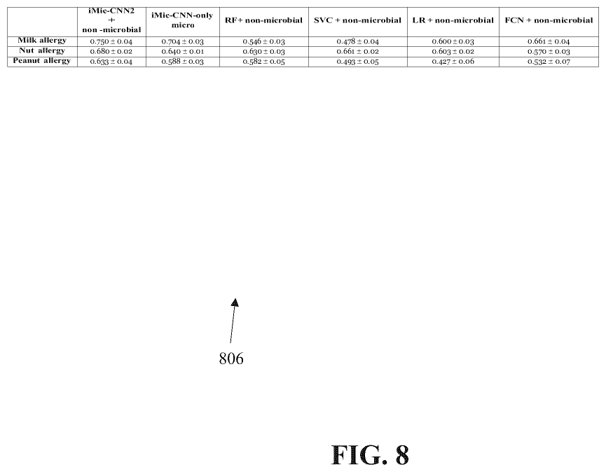

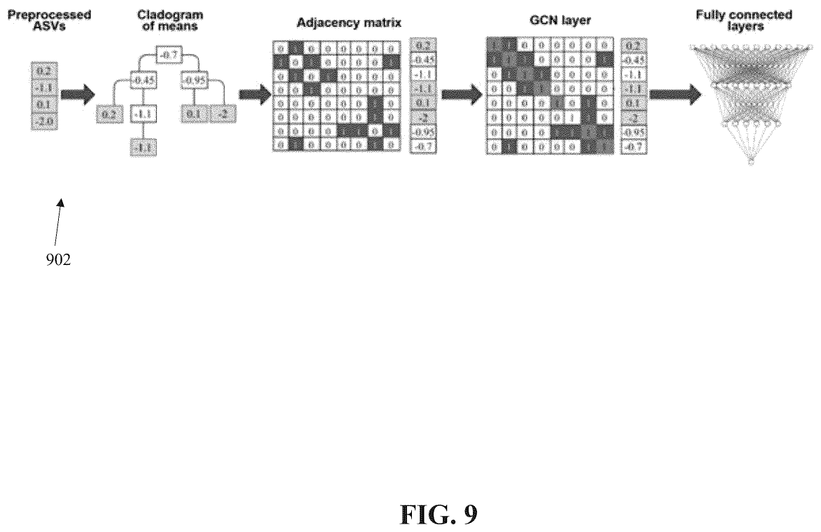

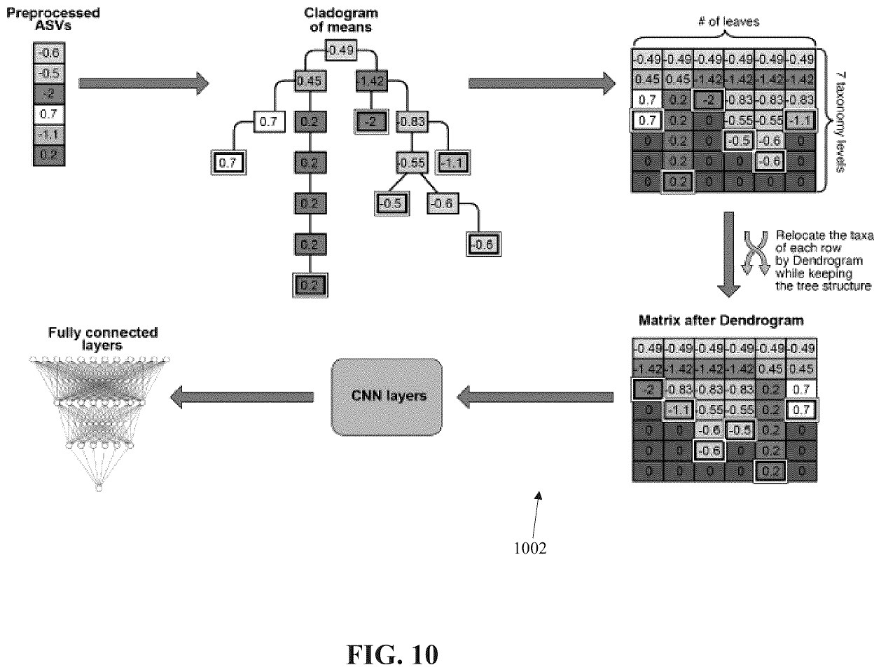

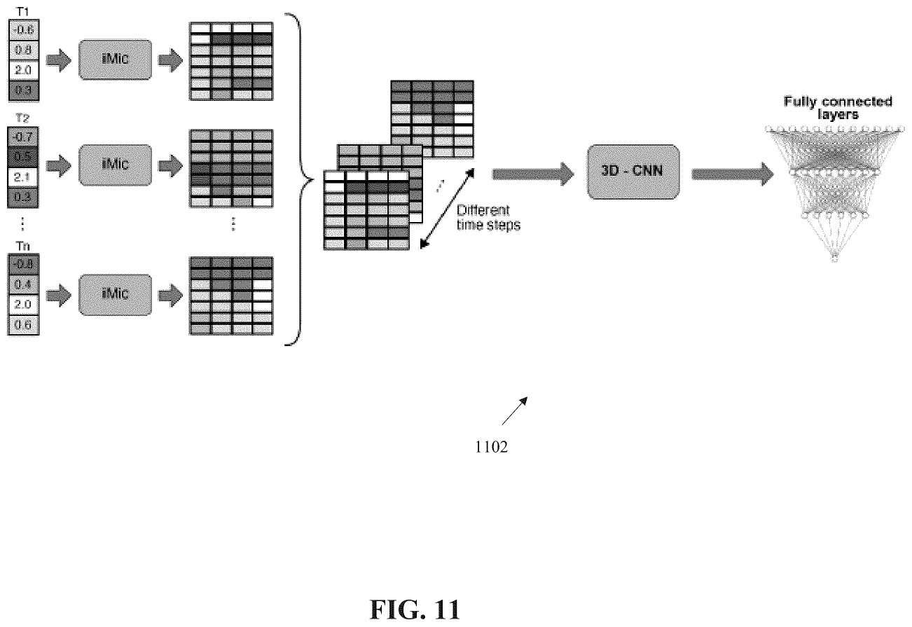

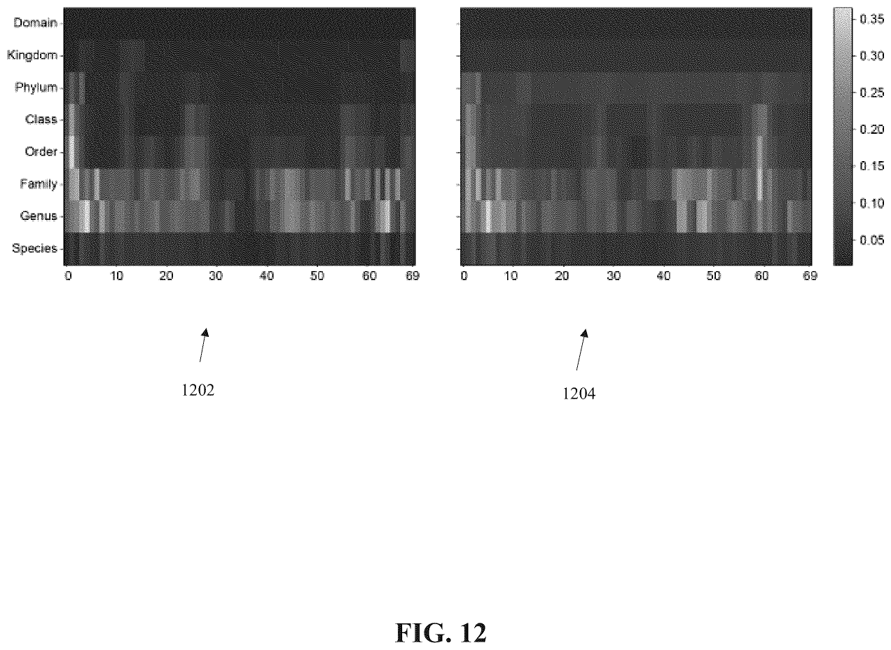

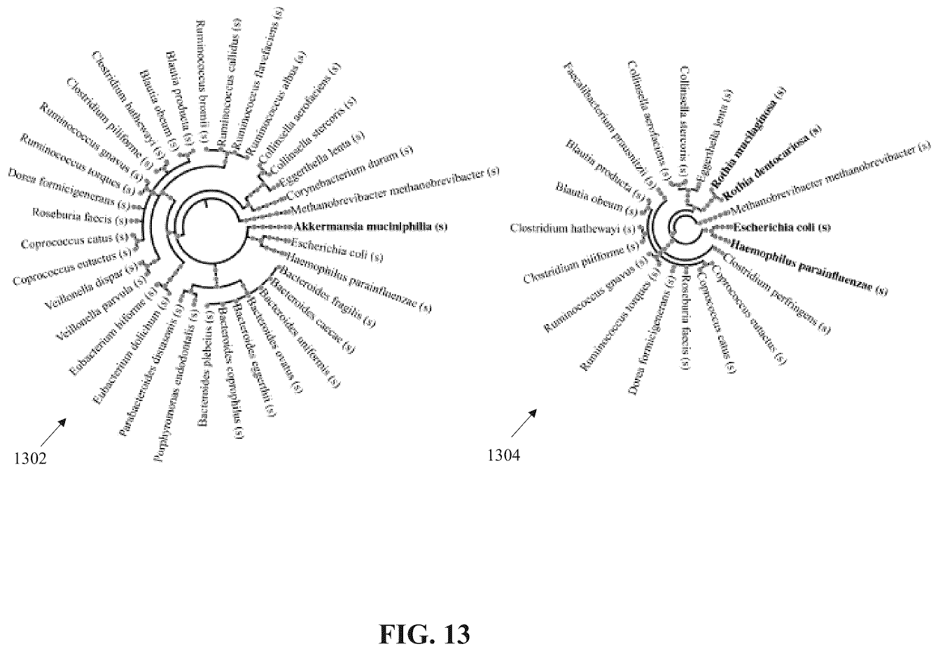

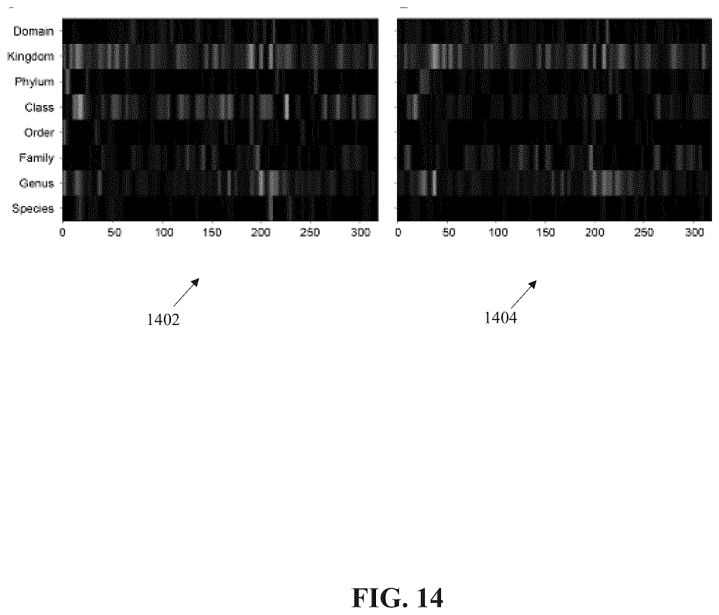

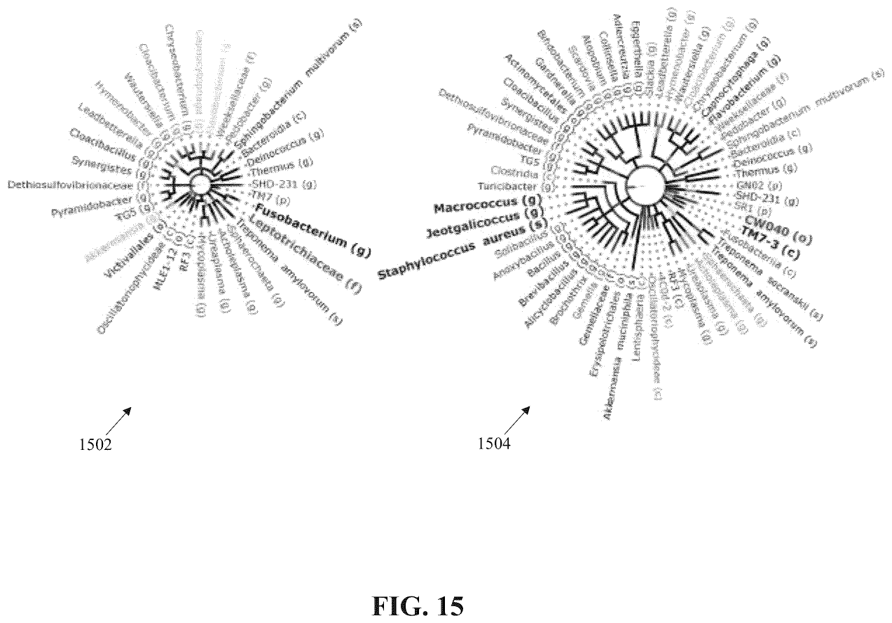

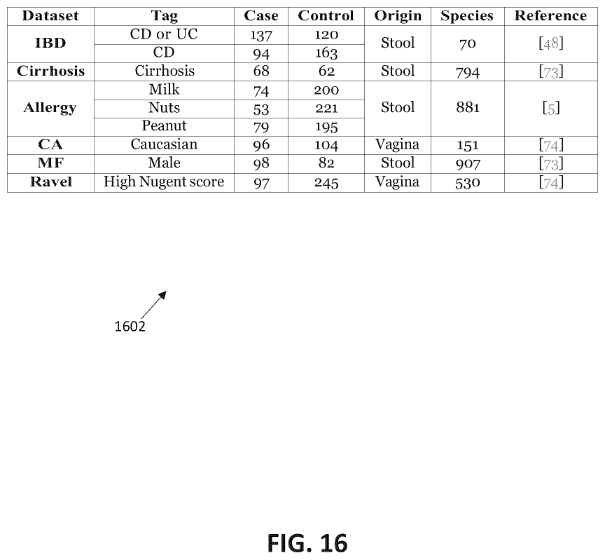

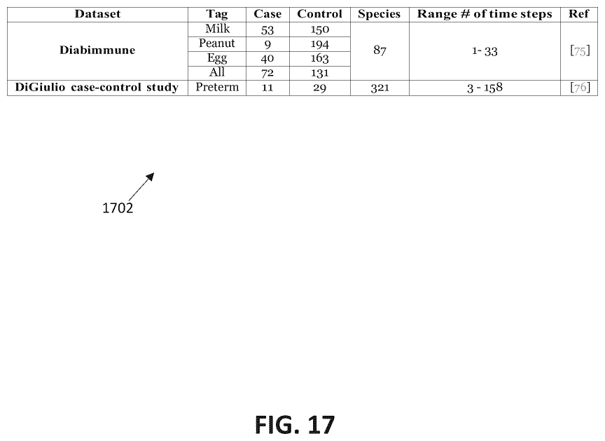

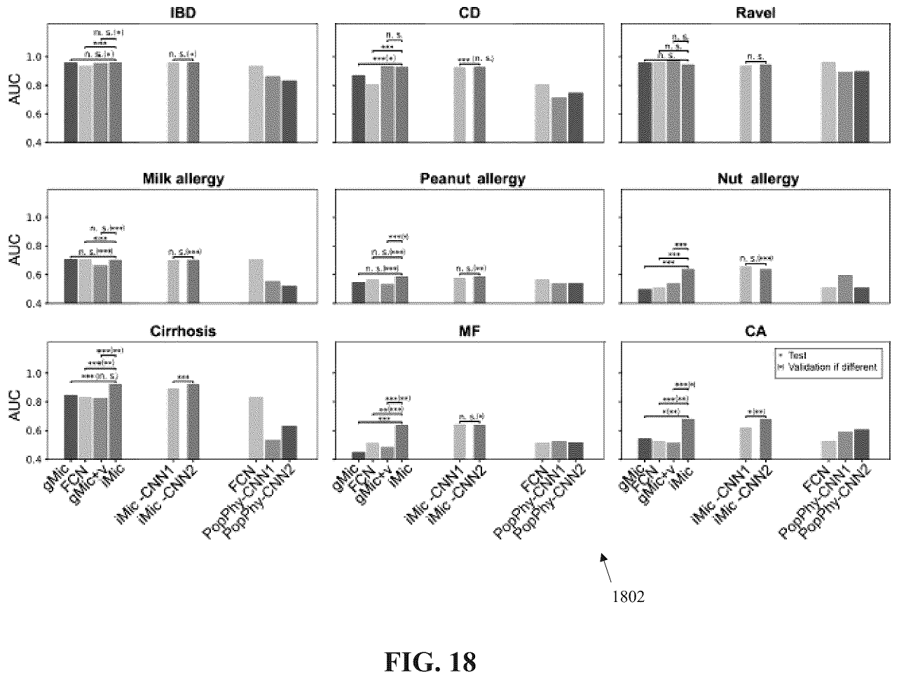

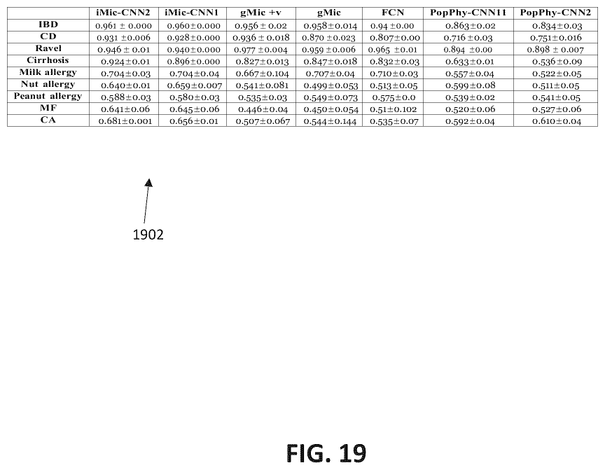

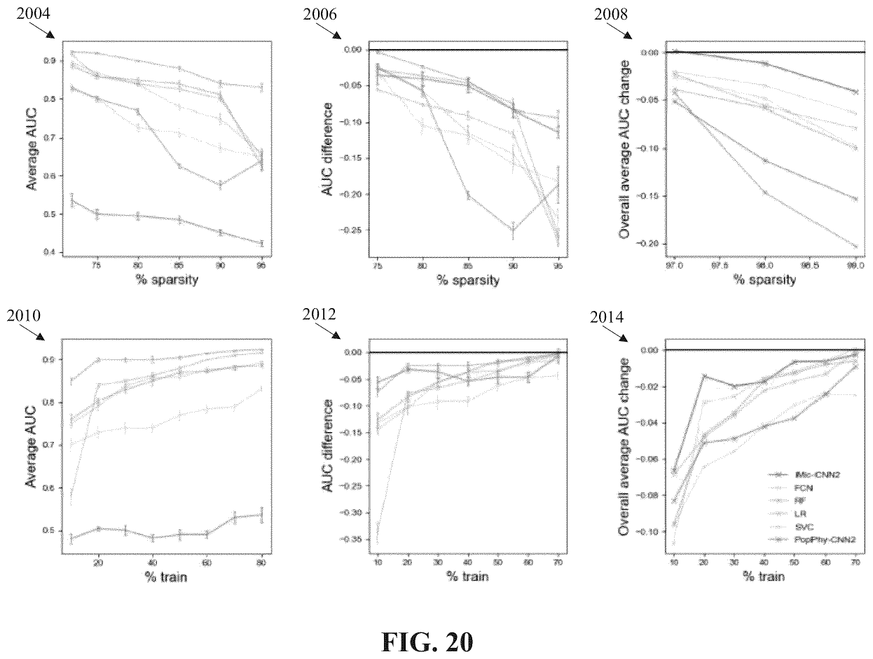

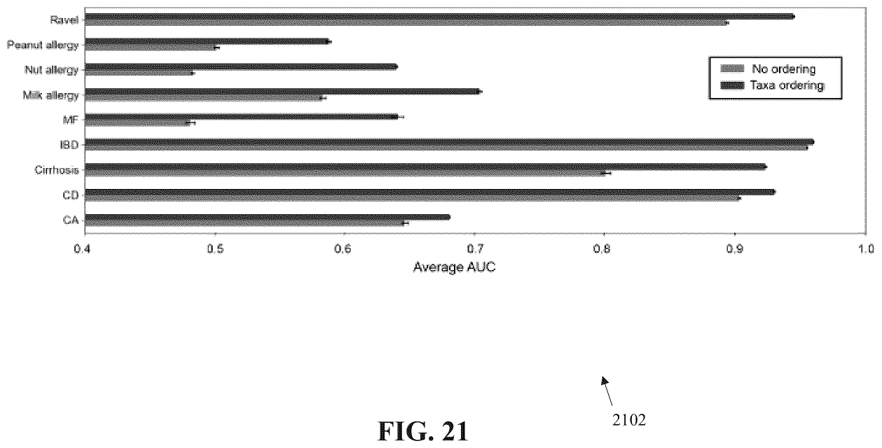

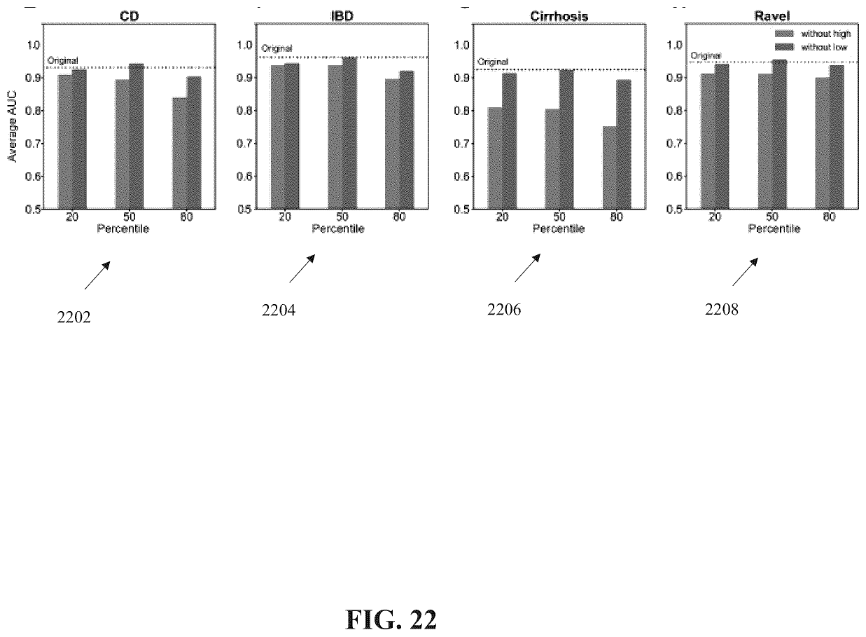

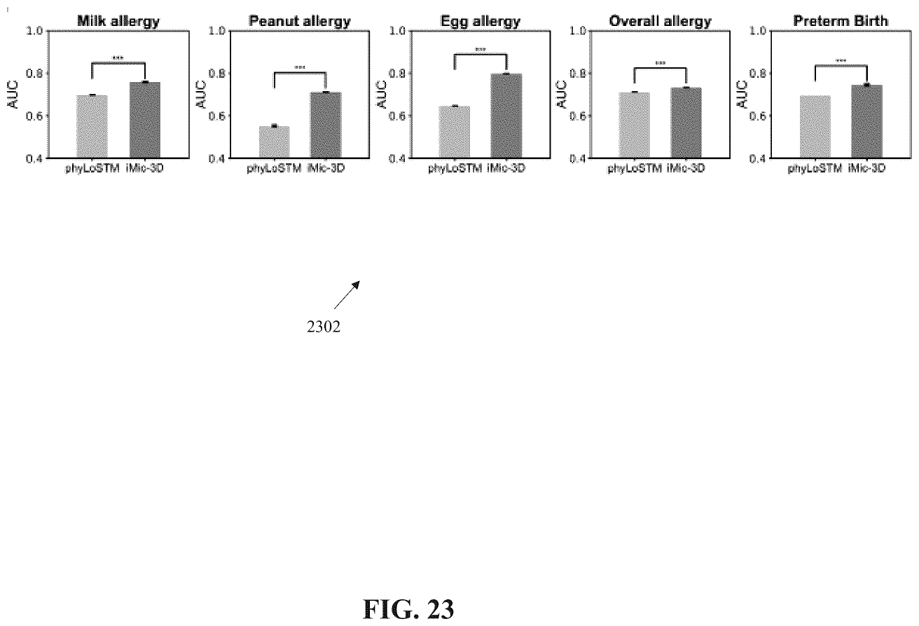

Reference is made to FIG. 1, which is a block diagram of a system 100 for computing a medical state of a subject based on a cladogram created from genetic sequences of a sample of a microbiome and/or training an ML model for computing the medical state of the subject based on the cladogram, in accordance with some embodiments of the present invention. Reference is also made to FIG. 2, which is a flowchart of an exemplary method of computing a medical state of a subject based on a cladogram created from genetic sequences of a sample of a microbiome, in accordance with some embodiments of the present invention. Reference is also made to FIG. 3, which is a flowchart of an exemplary method of training an ML model for computing a medical state of a subject based on a cladogram created from genetic sequences of a sample of a microbiome, in accordance with some embodiments of the present invention. Reference is also made to FIG. 4, which is a table 199 presenting comparison between existing methods' ability to address technical problems described herein, in accordance with some embodiments of the present invention. Reference is also made to FIG. 5, which is a pseudocode 502 of an exemplary approach for populating a mean cladogram, in accordance with some embodiments of the present invention. Reference is also made to FIG. 6, which is a pseudocode 602 of an exemplary approach for mapping the cladogram to a matrix, in accordance with some embodiments of the present invention. Reference is also made to FIG. 7, which is a pseudocode 702 of an exemplary reordering approach, in accordance with some embodiments of the present invention. Reference is also made to FIG. 8, which is a table 802 of examples of additional features that may be fed in combination with the image into the ML model, in accordance with some embodiments of the present invention. Reference is also made to FIG. 9, which is a schematic of an exemplary dataflow 902 of gMic+v, in accordance with some embodiments of the present invention. Reference is also made to FIG. 10, which is a schematic of an exemplary dataflow 1002 of iMic, in accordance with some embodiments of the present invention. Reference is also made to FIG. 11, which is a schematic 1102 of an exemplary dataflow of a 3D implementation of iMic, in accordance with some embodiments of the present invention. Reference is also made to FIG. 12, which includes Grad-Cam images 1202 and 1204, in accordance with some embodiments of the present invention. Reference is also made to FIG. 13, which includes cladogram projections 1302 and 1304, in accordance with some embodiments of the present invention. Reference is also made to FIG. 14, which includes heatmaps 1402 and 1404 created from three images, in accordance with some embodiments of the present invention. Reference is also made to FIG. 15, which includes Grad-Cam projections 1502 1504 of heatmaps 1402 and 1402 described with reference to FIG. 14 on the cladogram, in accordance with some embodiments of the present invention. Reference is also made to FIG. 16, which is a table 1602 of datasets used in the experiment, in accordance with some embodiments of the present invention. Reference is also made to FIG. 17, which is a table 1702 of the sequential datasets used in the experiment, in accordance with some embodiments of the present invention. Reference is also made to FIG. 18, which is a graph 1802 presenting a comparison between model performance performed as part of the Experiment described herein, in accordance with some embodiments of the present invention. Reference is also made to FIG. 19, which is a table 1902 of 10 CV mean performances based on experimental results, in accordance with some embodiments of the present invention. Reference is also made to FIG. 20, which includes graphs 2004-2014 indicating that iMic copes with the ML challenges above better than other methods based on experimental results, in accordance with some embodiments of the present invention. Reference is also made to FIG. 21, which includes graphs 2102 indicating importance of ordering taxa based on experimental results, in accordance with some embodiments of the present invention. Reference is also made to FIG. 22, which includes graphs 2202, 2204, 2206, and 2208 depicting interpretation based on experimental results, in accordance with some embodiments of the present invention. Reference is also made to FIG. 23, which is includes graphs 2302 comparing performance of 3D learning vs PhyLoSTM based on experimental results, in accordance with some embodiments of the present invention.

System 100 may implement the acts of the method described with reference to FIGS. 2-3 and/or other methods described herein, by processor(s) 102 of a computing device 104 executing code instructions 106A stored in a storage device 106 (also referred to as a memory and/or program store).

Computing device 104 may be implemented as, for example, a client terminal, a server, a single computer, a group of computers, a computing cloud, a virtual server, a virtual machine, a mobile device, a desktop computer, a thin client, a Smartphone, a Tablet computer, a laptop computer, a wearable computer, glasses computer, and a watch computer.

Multiple architectures of system 100 based on computing device 104 may be implemented. In an exemplary implementation of a centralized architecture, computing device 104 storing code 106A, may be implemented as one or more servers (e.g., network server, web server, a computing cloud, a virtual server) that provides services (e.g., one or more of the acts described with reference to FIGS. 2-3 and/or other methods described herein) to one or more client terminals 112 over a network 114, for example, providing software as a service (SaaS) to the client terminal(s) 112, providing software services accessible using a software interface (e.g., application programming interface (API), software development kit (SDK)), providing an application for local download to the client terminal(s) 112, and/or providing functions using a remote access session to the client terminals 112, such as through a web browser. For example, computing device 104 accesses a sequence dataset 106A of genetic sequences of a microbiome sample of a subject, computes a tree (e.g., cladogram), maps the tree to a data structure (e.g., image and/or graph), which is used to generate an ML model training dataset 106B for generating a trained ML model 106C, and/or which is fed into trained ML model 106C, as described herein. Multiple users use their respective client terminals 112 to access computing device 104, which may be remotely located. Each client terminal 112 may provide its own input data 124 for feeding into the trained ML model 124 running on computing device 104, for example, via the API, and/or via an application locally installed on client terminal 112, and/or by another file transfer protocol. Computing device 104 centrally processes genetic sequences 124 of microbes in a microbiome sample obtained from a respective subject, which may be received from each client terminal 112, by computing a tree and mapping the tree to the data structure which is fed into trained ML model 116C to generate an outcome, for example, state of a medical condition, as described herein. Computing device 104 may provide the outcome of trained ML model 106C to respective client terminal 112 (corresponding to each data 124) for example, for presentation on a display associated with client terminal 112 and/or for storage in an electronic health record.

In another example of a localized architecture, computing device 104 may include locally stored software (e.g., code 106A) that performs one or more of the acts described with reference to FIGS. 2-3 and/or other methods described herein, for example, as a self-contained system such as a laboratory server in communication with sequencing device 122. Code 106A may be implemented as a plug-in and/or additional feature set for integration with existing software that controls sequencing device 122. For example, a microbiome sample is sequenced by sequencing device 122, and genetic sequences 124 are obtained. A tree is computed from genetic sequences 124, which is mapped to a data structure, and fed into ML model 116C for obtaining the outcome, which may be presented on a display (e.g., 108) of computing device 104.

ML model training dataset 116B may be centrally and/or locally created based on sequences obtained from one or more sequencing devices 122. ML model 116C may be centrally and/or locally trained based on the centrally and/or locally trained ML model training dataset 116B. For example, a central ML model training dataset 116B is created from different microbiome samples obtained from different sample subjects which are sequenced at different sequencing devices 122, for example, in different cities, countries, and the like. A general trained ML model 116C may be created by training on the ML model training dataset 116B. In another example, specialized and/or personalized ML model training datasets 116B are created, for example, per anatomical location of microbiome sample (e.g., blood, stool, spit, vaginal), and/or per patient population (e.g., elderly, pregnant women, children), and/or per geographical location (e.g., healthcare facility, city). Respective specialized and/or personalized ML models 116C may be created by training on respective specialized and/or personalized ML models training datasets 116B.

Processor(s) 102 of computing device 104 may be implemented, for example, as a central processing unit(s) (CPU), a graphics processing unit(s) (GPU), field programmable gate array(s) (FPGA), digital signal processor(s) (DSP), and application specific integrated circuit(s) (ASIC). Processor(s) 102 may include multiple processors (homogenous or heterogeneous) arranged for parallel processing, as clusters and/or as one or more multi core processing devices. Processor(s) 102 may be arranged as a distributed processing architecture, for example, in a computing cloud, and/or using multiple computing devices. Processor(s) 102 may include a single processor, where optionally, the single processor may be virtualized into multiple virtual processors for parallel processing, as described herein.

Data storage device 106 stores code instructions executable by processor(s) 102, for example, a random access memory (RAM), read-only memory (ROM), and/or a storage device, for example, non-volatile memory, magnetic media, semiconductor memory devices, hard drive, removable storage, and optical media (e.g., DVD, CD-ROM). Storage device 106 stores code 106A that implements one or more features and/or acts of the method described with reference to FIGS. 2-3 and/or other methods described herein when executed by processor(s) 102.

Computing device 104 may include a data repository 116 for storing data, for example, storing one or more of a sequence dataset 116A which include genetic sequences of microbiome samples of subjects, ML model training dataset 116B created as described herein, and/or trained ML model 116C created as described with reference to FIG. 3 and/or used as described with reference to FIG. 2, cladogram repository 116D that includes the cladogram computed from genetic sequences of microbiome samples, and/or data structure repository 116E that includes the image and/or graph computed by mapping the cladogram, as described herein. Data repository 116 may be implemented as, for example, a memory, a local hard-drive, virtual storage, a removable storage unit, an optical disk, a storage device, and/or as a remote server and/or computing cloud (e.g., accessed using a network connection).

Computing device 104 may include a network interface 118 for connecting to network 114, for example, one or more of, a network interface card, a wireless interface to connect to a wireless network, a physical interface for connecting to a cable for network connectivity, a virtual interface implemented in software, network communication software providing higher layers of network connectivity, and/or other implementations.

Network 114 may be implemented as, for example, the internet, a local area network, a virtual private network, a wireless network, a cellular network, a local bus, a point to point link (e.g., wired), and/or combinations of the aforementioned.

Computing device 104 may connect using network 114 (or another communication channel, such as through a direct link (e.g., cable, wireless) and/or indirect link (e.g., via an intermediary computing unit such as a server, and/or via a storage device) with one or more of:
Server(s) 120 storing one or more dataset(s) 120A, for example, sequence dataset 116A, as described herein.
Sequencing device 122 that sequences samples of microbes of a microbiomes, for example, generates genetic sequences of the 16S rRNA of multiple microbes from a sample of a microbiome, as described herein.
Client terminals 112, which may provide data for input 124 into trained ML model 116C, as described herein.

Computing device 104 and/or client terminal(s) 112 include and/or are in communication with one or more physical user interfaces 108 that include a mechanism for a user to enter data (e.g., provide the data 124 for input into trained ML model 116C) and/or view the displayed outcome of ML model 116C, optionally within a GUI. Exemplary user interfaces 108 include, for example, one or more of, a touchscreen, a display, a keyboard, a mouse, and voice activated software using speakers and microphone.

At 202, one or more ML models are trained and/or accessed. The machine learning model processes neighboring representations based on their relative positions, for example, the kernel of the CNN processes pixels of the image that are closer together differently than pixels of the image that are further apart.

There may be different ML models. For example, different architectures based on different implementations of the data structure, for example, a CNN for an image, and a GNN for a graph. In another example, the different ML models are for microbiome samples obtained from different anatomical locations in a subject, for example, stool, vagina, sputum, blood, and the like. In another example, the different ML models are for different states of medical conditions. In yet another example, the different ML models are for different types of subject, for example, different demographics, such as pregnant women, peoples age 30-50 with Crohn's Disease, and the like.

An exemplary approach for training the ML model(s) is described, for example, with reference to FIG. 3.

Exemplary architectures are described herein.

At 204, genetic sequences of microbes of one or more microbiome samples of a subject are obtained.

The microbiome sample(s) may be obtained from an anatomical location and/or from body substances, for example, stool, blood, sputum, rectum, mouth, and vagina.

Multiple sequential microbiome samples may be obtained from the same subject, for example, from a similar anatomical location and/or similar body substance spaced apart in time (e.g., a few minutes, a day, a week, a month, a year, and the like). In another example, multiple microbiome samples may be obtained from different anatomical locations and/or different body substances, at spaced part time intervals. The multiple microbiome samples, which corresponding to the multiple spaced apart time intervals, may be used to generate a temporal 3D data structure (e.g., 3D image) which is fed into a 3D ML model, for example, as described herein.

At 206, the genetic sequences may be pre-processed to compute representations which are used to populate the cladogram.

Optionally, the pre-processing includes clustering the genetic sequences to create Amplicon Sequence Variants (ASVs). A vector of the ASVs may be created, where each entry of the vector represents a microbe at a certain taxonomy level of a taxonomy hierarchy. A log-normalization of the ASV frequencies of the vector may be computed, to obtain a log-normalized vector of ASV frequencies.

The cladogram may be created from the vector (e.g., as described with reference to 208) by placing respective values of the vector, optionally the log-normalized values, at corresponding taxonomic category levels of the taxonomy hierarchy of the tree.

The taxonomy levels of the taxonomy hierarchy may be for example, Super-kingdom, Phylum, Class, Order, Family, Genus, and Species.

Additional exemplary details of pre-processing are now described.

The 16S rRNA gene sequence of the microbiome samples may be processed via the MIPMLP pipeline [46].The preprocessing of the MIPMLP pipeline includes 4 stages: merging similar features based on the taxonomy, scaling the distribution, standardization to z-scores, and dimension reduction.

The features of the species taxonomy may be merged, for example, using the Sub-PCA method that performs a PCA (Principal component analysis) projection on each group of microbes in the same branch of the cladogram. Log normalization and/or z-scoring may be performed. Dimension reduction may not necessarily be performed at this stage. When species classification is unknown, the best-known taxonomy may be used.

At 208, a tree that includes the representations of the genetic sequences of the microbes of the microbiome sample (e.g., created as described with reference to 206) may be created.

The tree includes the representations of the microbes arranged according to a taxonomy hierarchy, for example, each log-normalized ASV is placed at the taxonomic level to which the log-normalized ASV is mapped.

The tree may be created by including each observed taxa of microbes of the microbiome sample in a leaf at a respective taxonomic level of the taxonomy hierarchy. The leaves are the preprocessed observed samples (each at its appropriate taxonomic level). The log-normalized frequency of the observed taxa of microbes may be added to each leaf. Each internal node includes an average of direct descendants of the internal node located at lower levels. The internal vertices of the cladogram may be populated with the average over their direct descendants at a finer level (e.g., for the family level, an average over all genera belonging to the same family is computed).

The iMic framework may include 3 exemplary not necessarily limiting processes, which are described herein in additional detail: Populating the mean cladogram, Cladogram2Matrix (e.g., as described with reference to 210A), and feed into a ML model (e.g., as described with reference to 214A).

Given a vector of log-normalized ASVs frequencies merged to taxonomy 7-b, each entry of the vector, bi represents a microbe at a certain taxonomic level. An average cladogram may be built, where each internal node is the average of its direct children. Once the cladogram is populated, A representation matrix may be built.

Notation used here are summarized in Table 1 below:

TABLE 1
Notations

| | |
|---|---|
| b | ASVs preprocessed vector (only the leaves of the cladogram) |
| R | Raw representation matrix |
| \hat{R} | Rearranged representation matrix |
| l | A taxonomy level (Super-kingdom, Phylum, Class, Order, |
| | Family, Genus, Species) |
| N | Number of leaves of the cladogram of means |
| C_{l, i} | Hierarchical cluster number i in level l |
| A | Adjacency matrix of graph |
| σ | Activation function |
| W | Weight matrix in neural network |
| I | Identity matrix |
| v | ASV frequency vector (all the cladogram's vertices, and not |
| | only the leaves, in contrast with b above) |

Referring now back to FIG. 5, pseudocode 502 of the exemplary approach for populating the mean cladogram, is presented.

Referring now back to FIG. 2, at 210, the tree is mapped to a data structure. The data structure may define the taxonomy hierarchy and/or define positions between neighboring representations of the microbes. The data structure may be an image and/or graph, where the location of each element (e.g., pixel, node) is define with respect to the location of other elements. Changing the relative position of the elements in the data structure may impact the information content and/or the processing of the data structure. This is in contrast to the tree, where the position between neighboring elements at the same taxonomy hierarchy is arbitrary, and are interchangeable without impacting the information content and/or processing of the tree. Optionally, the data structure is implemented as a graph. The graph may include values of the tree and an adjacent matrix.

Alternatively or additionally, the data structure is represented as an image. The image may be created by projecting the tree to a two dimensional matrix. The 2D matrix includes multiple of rows matching a number of different taxonomy category levels of the taxonomy hierarchy represented by the tree, (e.g., 7 or 8), and a column for each leaf.

Optionally, at each respective level of the different taxonomy category levels, a value of the two dimensional matrix at the respective level is set to the value of leaves below the level. A value at a higher level than the respective level may be set to have an average of values of a level below the respective level. Below a leaf values are set to zero. When a level below the respective level includes multiple different values, multiple positions of the two dimensional matrix of the current respective level are set to an average of the different values of the level below the respective level.

Exemplary approaches for mapping the cladogram to an image, part of the iMic framework, are now described with reference to 210A. Exemplary approaches for mapping the cladogram to a graph, part of the gMic framework, are now described with reference to 210B.

Referring now back to FIG. 2, at 210A, the image (e.g., used in iMic) may be initiated with the same tree used for the graph (e.g., used in gMic), but then instead of a GCN, the cladogram with the means in the vertices is projected to a two-dimensional matrix with rows according to the number of taxonomic levels in the cladogram (e.g., 7 or 8), and a column for each leaf.

Each leaf may be set to its preprocessed frequency at the appropriate level and zero at the finer level (e.g., for an ASV at the genus level, the species level of the same genus is 0). Values at a coarser level (e.g., at the family level) are the average values of the level below (i.e., a finer level-genus). For example, if there are 3 genera belonging to the same family, at the family level, the 3 columns will receive the average of the 3 values at the genus level.

In terms of mathematical representation, a matrix R ∈R8×N is created, where N denotes the number of leaves in the cladogram and 8 denotes the 8 taxonomic levels (or other number may be used, for example, 7), such that each row represents a taxonomic level. The values of the tree may be added layer by layer to the image, starting with the values of the leaves. If there are taxonomic levels below the leaf in the image, they may be populated with zeros. Above the leaves, for each taxonomic level the average of the values in the layer below may be computed. When the layer below has multiple (denoted k) different values, the average may be set to all k positions in the current layer. For example, if there are 3 species within one genus with values of 1,3 and 3. A value of 7/3 is set to the 3 positions at the genus level including these species.

The transformation of the microbiome sample into an image may be extended to translate a set of microbiome samples to a movie and/or 3D image and/or combined image, which to classify sequential microbiome samples. A 2-dimensional representation for the microbiome of each time step may be computed. The multiple 2D representations may be combined into a movie and/or 3D image and/or combined image of the microbiome samples. For example, the multiple 2D images are placed neighboring each other to create a third dimension. In another example, each 2D image is defined as a different channel (e.g., color) and a combined image is created by including the different channels in a single image (e.g., multi-colored). In yet another example, the 2D images are placed sequentially one after the other, to create a sequence of images, which may be termed a movie.

It is noted that while a graph (e.g., gMic) captures the relation between similar taxa, the graph does not necessarily solve the sparsity problem described herein. To solve the sparsity problem, iMic may be used for a different combination of the relation between the structure of the cladogram and the taxa's frequencies into an image and applying CNNs on this image to classify the samples.

Referring now back to FIG. 6, pseudocode 602 of the exemplary approach for mapping the cladogram to the 2D matrix (i.e., image), is presented.

Referring now back to FIG. 2, alternatively to 210A, at 210B, the data structure may be represented as a graph, for example, part of the gMic framework.

There may be different implementations of the graph of gMic. For example, in a simpler version, the microbial count is ignored. The frequencies of all existing taxa are replaced by a value of 1. The cladogram structure is used on its own. In another version, termed herein gMic+v, the normalized taxa frequency values are included.

In some embodiments, termed gMic+v, the cladogram and the gene frequency vector are used to create the graph. The graph may be represented by the symmetric normalized adjacency matrix, denoted A^{˜}, for example, in the following equations:

$${A}^{∼} = {D}^{- 1 / 2} {AD}^{- 1 / 2} \qquad Equation ( 3 )$$

$$D is a diagonal matrix such that {D}_{ii} = {\sum }_{j} {A}_{ij} \qquad Equation ( 4 )$$

Alternatively in other embodiments termed gMic, the cladogram is built and populated as in iMic. In gMic, a GCN layer may be applied to the cladogram. The output of the GCN layer is the input to a fully connected neural network (FCN) (e.g., as in 214B) for example:

$$σ ( ( {A}^{∼} + α \cdot I ) \cdot sign ( v ) \cdot W ) FCN \qquad Equation ( 5 )$$

where: v denotes the ASVs frequency vector (all the cladogram's vertices, and not only the leaves, in contrast with b above), sign(v) denotes the same vector where all positive values were replaced by 1 in the gene frequency vector (i.e., the values are ignored). a denotes a learned parameter, regulating the importance given to the vertices' values against the first neighbors, W denotes the weight matrix in the neural network, and a denotes the activation function.

At 212, elements of the data structure are re-ordered. Each element, for example, a pixel of the image and/or node of the graph, denotes a type of microbe at a certain taxonomic level of the taxonomy hierarchy. The re-ordering may be done for the image and/or for the graph.

The re-ordering may be done according to one or more similarity features between different types of microbes at the same taxonomy category level of the taxonomy hierarchy while preserving the structure of the tree (which was mapped to the data structure). The ordering may be done per taxonomic category level, for positioning the representations of microbes that are more similar closer together and representation of microbes that are less similar are positioned further apart while preserving the structure of the tree. The ordering may be done for positioning taxa with similar frequencies relatively closer together and taxa with less similar frequencies further apart.

Optionally, the similarity feature used for the re-ordering is based on similarity of frequencies of the microbes.

Optionally, the similarity feature may be computed based on Euclidean distances between representation, optionally Euclidean distances between the frequencies, optionally the log-normalized frequencies.

Optionally, the similarity feature may be computed by building a dendrogram based on Euclidean distances computed for a hierarchical clustering of the representations of the microbes.

The ordering may be computed recursively per taxonomic category level, optionally starting from the lowest level towards higher levels. In embodiments in which the ASV frequencies are log-normalized, the recursive ordering may be of the log-normalized ASV frequencies according to the similarity feature(s) computed based on Euclidean distances between log-normalized ASV frequencies.

Exemplary approaches for re-ordering the image are now described.

Columns of the image may be sorted recursively, so that taxa with more similar frequencies in the dataset are closer using hierarchical clustering on the frequencies within a subgroup of taxa. For example, assume 3 sister taxa, taxona, taxonb, and taxonc, the order of those 3 taxa in the row (e.g., at a same taxonomy category level) may be determined by their proximity in a Dendrogram generated based on their frequencies.

The microbes may be re-ordered at each taxonomic level (row) such that similar microbes are close to each other in the produced image. Optionally, a dendrogram based on the Euclidean distances may be used as a similarity feature metric using complete linkage on the columns, relocating the microbes according to the new order while keeping the phylogenetic structure. The order of the microbes may be created recursively. The recursive re-ordering may be performed for example as follows: reordering the microbes on the phylum level, relocating the phylum values with all their sub-tree values in the matrix. Then a dendrogram of the descendants of each phylum is built separately, reordering them and their sub-tree in the matrix. The recursive reordering is iterated until all the microbes in the species taxonomy of each phylum are ordered.

Referring now back to FIG. 7, pseudocode 702 of the exemplary approach for reordering, is presented.

Referring now back to FIG. 2, at 214, the data structure is fed into a machine learning model.

The implementation of the ML model(s) may be according to the type of the data structure, for example, a CNN for an image, and a GNN for a graph.

The machine learning model may process neighboring representations based on their relative positions, for example, the kernel of the CNN processes pixels of the image that are closer together differently than pixels of the image that are further apart.

An exemplary implementation of the ML model designed to process images is described with reference to 214A. An exemplary implementation of the ML model designed to process graphs is described with reference to 214B.

Referring now back to FIG. 2, at 214A, the image may be fed into a CNN, or other ML model designed to process images, such as other neural network architectures.

Optionally, non-microbial features may be fed into the ML model (e.g., of iMic), for example, by concatenating the non-microbial features to the flattened microbial output of the last CNN layer before the fully connected (FCN) layers. Other approaches of feeding the non-microbial features into the CNN in combination with the image may be used.

Examples of non-microbial features include: sex, HDM, atopic dermatitis, asthma, age, dose of allergen, and/or indication of other demographic parameters of the subject and/or other medical history of the subject.

Referring now back to FIG. 8, table 802 presents features that may be added to iMic's learning in combination with images, and/or fed in into the ML model in combination with image(s). Average AUCs of iMic-CNN2 with and without non-microbial features as well as average results of naive models with non-microbial features were obtained from the experiment described in the Examples section. The results are the average AUCs on an external test with 10 CVs±their standard deviations (stds) obtained as part of the experiment described in the Examples section. The results indicate that adding non-microbial features even further improves the results of iMic (e.g., higher accuracy) when compared to a model without non-microbial features.

Optionally, for the case of multiple microbiome samples, multiple trees are computed for the microbiome samples. The trees are mapped to multiple data structures (e.g., image representations). The images are combined into a 3D image. The 3D image is fed into a 3D-CNN implementation of the machine learning model.

Alternatively or additionally to 214A, at 214B, when the data structure is implemented as a graph, the tree and adjacent matrix representing the graph may be fed into a layer of a graph convolutional neural network implementation of the machine learning model. An identity matrix with a learned coefficient may be added. Output of the layer may be fed into a fully connected layer that generates the outcome.

The graph may be used as the convolution kernel of a GCN, followed by for example two fully-connected layers, to predict the class of the sample.

The non-microbial features may be fed into the GCN in combination with the graph.

Referring now back to FIG. 9, the exemplary dataflow 902 of gMic+v (e.g., architecture) is presented. The exemplary dataflow 902 may be based on features 208, 210B, and 214B described with reference to FIG. 2, and/or or features described herein. The dataflow includes the following exemplary steps: positioning observed taxa (e.g., all observed taxa) in the leaves of the taxonomy tree (cladogram), and setting their value to the preprocessed frequency to each leaf. Each internal node is the average of its direct descendants. These values are the input to a GCN layer with the adjacency matrix of the cladogram. The GCN layer is followed by two fully connected layers with binary output.

Referring now back to FIG. 10, the exemplary dataflow 1002 of iMic is presented. The exemplary dataflow 1002 may be based on features 208, 210A, 212, and 214A described with reference to FIG. 2, and/or or features described herein. The values in the cladogram are as in gMic+v. The cladogram is then used to populate a 2-dimensional matrix. Each row in the image represents a taxonomic level. The order in each row is based on a recursive hierarchical clustering of the sample values preserving the structure of the tree. The image is the input of a CNN followed by 2 fully connected layers with binary output.

Referring now back to FIG. 11, schematic 1102 depicts the exemplary dataflow of the 3D implementation of iMic, for 3D learning. The ASV frequencies of each snapshot are preprocessed and combined into images as in the static iMic. The images from the different time points are combined into a 3D image, which is the input of a 3-dimensional CNN followed by two fully connected layers that return the predicted phenotype.

At 216, the state of the medical condition of the subject is obtained as an outcome of the ML model. The state of the medical condition may be, for example, presented on a display, stored on a data storage device (e.g., of a server and/or computer) such as within an electronic health record of the subject, printed (e.g., in a report for the subject), and/or fed into another process (e.g., as described with reference to 218 of FIG. 2).

The state of the medical condition may be binary, for example, whether the subject has the medical condition or does not have the medical condition. Examples of medical conditions include: inflammatory bowel disease (IBD) such as Crohn's Disease (CD) and/or Ulcerative Colitis (UC), cirrhosis, allergy (e.g., to mil, nuts, peanuts), and the like. The state may be a classification category, for example, a subtype of the medical condition (e.g., CD or UC), and/or intensity of the medical disease.

Optionally, the state of the medical condition may be an indication of complication(s) that arise during pregnancy, which may be linked to lifelong health risks, for example, gestational diabetes which is linked to lifelong type 2 diabetes, preeclampsia which is linked to future cardiovascular disease, preterm birth which is linked to increased risk for mental and health conditions of the mother and/or fetus, and postpartum depression which is linked to mental health challenges.

At 218, an explainable artificial intelligent (AI) platform may be applied to the machine learning model for obtaining an estimate of portions of the data structure (e.g., image, graph) used by the machine learning model for obtaining the outcome. Examples of explainable AI platforms include: SHAP (SHapley Additive exPlanations), Lime (Local Interpretable Model-Agnostic Explanations), and Grad-Cam (Gradient-weighted Class Activation Mapping) and/or other platforms for generating heatmaps based on the input image. The portions of the data structure (e.g., image, data structure) identified by the explainable AI platform may be projected to the tree for obtaining an indication of which microorganisms of the microbiome most contributed to the outcome of the machine learning model. The patient may then be treated according to the identified microorganisms, for example, by antibiotics, pro-biotics, and/or other approaches, for example, as described with reference to 220 of FIG. 2.

Optionally, for the case of multiple microbiome samples (e.g., used to create the 3D image), the administration of the explainable AI platform may be performed for each of the data structures of the (e.g., sequentially) obtained microbiome samples. For example, creating multiple heatmaps each at a different channel (e.g., color). The multiple heatmaps may be combined into a single multi-channel image, for example, multi-color image. The multi-channel image may be projected on the tree to identify the most significant microorganisms.

Additional exemplary details are now described. iMic may be used to detect the taxa most associated with the state of the medical condition predicted by the ML model. For example, Grad-Cam (an explainable AI platform [53]) may be used to estimate the part of the image used by the ML model to classify each class [54]. In an exemplar implementation, the gradient information flowing into the first layer of the CNN is estimated to assign importance. The importance of the pixels may be averaged for control and case groups separately. It is noted that based on the experiments described herein, the CNN is most affected by the family and genus level (fifth row and sixth row in FIG. 12). The CNN uses different taxa for the case and the control (see FIG. 12). To find the microbes that most contributed to the classification, the computed Grad-Cam values are projected back to the cladogram (e.g., see 102 and 104 of FIG. 13).

To understand what temporal features of the microbiome are used for the classification, the heatmap of backwards gradients of each time step may be calculated separately, for example, using Grad-Cam. CNNs with a window of 3-time points may be used (or other number of samples may be used0. The heatmap of the contribution of each pixel in each time step may be represented as a channel for example, in the R, G, and B color space (or other channels may be used). An image that combines the cladogram and time effects may be created. The generated image may be projected on the cladogram.

Referring now back to FIG. 12, grad-Cam images 1202 and 1204 are presented. Each image 1202 1204 represents the average contribution of each input value to the gradients of the neural network back-propagation, as computed by the Grad-Cam algorithm. The Grad-Cam was placed after the first CNN layer. The results presented here are from the CD dataset of the experiment described in the Examples section. Image 1202 represents the average gradients for the healthy subjects of the cohort and image 1204 represents the average gradients for the CD subjects. The color reflects the average values of the gradients, such that the darker (e.g., blue) colors represent low gradients, and the lighter (e.g., yellow) colors represent the high gradients, using the ‘viridis’ colormap. The differences between the two heatmaps 1202 and 1204 represent the contribution of different taxa to the prediction of different phenotypes. Note that the main contribution to the classification is at the genus and family level (rows 6 and 5). Similar results were obtained for the other datasets.

Referring now back to FIG. 13, cladogram projections 1302 and 1304 are presented, to visualize the taxa contributing to each class, where image 1302 is computed for the healthy class and image 1304 is computed for the class. The most significant microbes are projected back on the cladogram. The lighter colored (e.g., purple) points on the cladograms represent taxa that are in the top decile of the gradients. The taxa in bold are important taxa that are consistent with the literature.

Referring now back to FIG. 14, heatmaps 1402 and 1404 were created from three images. To visualize the three-dimensional gradients, a CNN with a time window of 3 (i.e., 3 consecutive images combined using convolution) was used. The Grad-Cam images are projected to the R, G, and B channels of an image. Each channel represents another time point where R=earliest, G=middle, and B=latest time point. C-D. In heatmaps N02 and N04, each pixel represents the value of the backpropagated gradients after the CNN layer. The 2-dimensional images 1402 and 1404 are the combination of the 3 channels, i.e., the gradients of the first/second/third time step are in red/green/blue. Left image 1402 is for normal birth subjects in the DiGiulio dataset, and right image 1404 is for pre-term birth subjects, of the datasets used in the Experiments.

Referring now back to FIG. 15, Grad-Cam projections 1502 1504 of heatmaps 1402 and 1402 described with reference to FIG. 14 projected on the cladogram, as described. The taxa in bold are important taxa that are consistent with the literature.

At 220, the subject may be treated according to the indication of the state of the medical condition and/or according to the results of the applied ML interpretability model such as the most significant microorganisms.

The treatment may be selected for being effective for the state of the medical condition. The treatment may be, for example, antibiotics, pro-biotics, surgery, preventive measures, diet plan, exercise, alternative therapies (e.g., massage, acupuncture), and the like. The treatment may be to do nothing and/or watchful waiting and/or close monitoring.

Optionally, one or more features described with reference to 204-220 may be iterated, for example, for the same patient over multiple time intervals (e.g., weekly, monthly, quarterly) to monitor change in the state of the medical condition, prior to treatment and after treatment (e.g., to monitor effect of the treatment), before and/or after a first treatment and before and/or after change to a different treatment (e.g., to monitor effect of the change), before and/or after a treatment and before and/or after stopping a treatment (e.g., to monitor effect of the stopping of the treatment).

Referring now back to FIG. 3, at 302, genetic sequences of microbes of multiple sample microbiome samples of multiple subjects are obtained, for example, as described with reference to 204 of FIG. 2.

At 304, the genetic sequences may be pre-processed, for example, to compute a log-normalized vector of ASV values, for example, as described with reference to 206 of FIG. 2. Optionally, the genetic sequences of the microbes of multiple sample microbiome samples of multiple subjects are pre-processed.

At 306, a cladogram that includes representations may be computed from the genetic sequences of the microbes of the multiple sample microbiome samples of multiple subjects, for example, as described with reference to 208 of FIG. 2. The tree includes the representations of the microbes arranged according to a taxonomy hierarchy.

At 308, the cladogram is mapped to a data structure that defines the taxonomy hierarchy and defines positions between neighboring representations of the microbes (e.g., per sample microbiome sample), for example, an image and/or graph, for example, as described with reference to 210 of FIG. 2.

At 310, the data structure, optionally the image, is re-ordered, for example, as described with reference to 212 of FIG. 2.

At 312, an indication of a state of a medical condition is obtained per subject, for example, by manual user entry (e.g., via a user interface), from an electronic health record of the subject, and the like. Exemplary states of medical conditions of subjects are described, for example, with reference to 216 of FIG. 2.

At 314, multiple records are created. Each record includes the data structure (e.g., cladogram), optionally ordered (e.g., as described with reference to 310 of FIG. 3), and a ground truth indication of the state of the medical condition of respective subject (e.g., as described with reference to 312 of FIG. 3). Optionally, each record is for a respective sample microbiome sample. The multiple records are for the multiple sample microbiome samples.

The multiple records are included in a training dataset.

Different training datasets may be created, which are used for training different ML models. For example, a training dataset may include microbiome samples obtained from similar anatomical locations in patient. Another training dataset may be for a similar types of medical condition of the subject. Yet another training dataset may be for a similar type of subject, for example, pregnant women, peoples age 30-50 with Crohn's Disease, and the like.

At 316, one or more ML model(s) are trained on the training dataset. The implementation of the ML model(s) may be according to the type of the data structure, for example, a CNN for an image, and a GNN for a graph. The machine learning model processes neighboring representations based on their relative positions, for example, the kernel of the CNN processes pixels of the image that are closer together differently than pixels of the image that are further apart. The trained machine learning model generates the state of the medical condition of the subject in response to an input of a data structure, optionally ordered, computed from a microbiome sample obtained from the subject.

The ML model may be trained by a transfer learning approach, for example, an existing pre-trained CNN trained on other types of images is then further training on the training dataset using a transfer learning approach.

Various embodiments and aspects of the present disclosure as delineated hereinabove and as claimed in the claims section below find experimental and/or calculated support in the following examples.

### EXAMPLES

Reference is now made to the following examples, which together with the above descriptions illustrate some embodiments in a not necessarily limiting fashion.

It is noted that some technical details described above are repeated below for completion of the description of the experiments performed by Inventors, and clarity of explanation.

Inventor's main hypothesis was that the cladogram of a microbiome sample is by itself an informative biomarker of the sample class, even when the frequency of each microbe is ignored. To test this, Inventors analyzed 6 datasets with 9 different phenotypes.

Referring now back to FIG. 16, table 1602 of datasets used in the experiment is presented.

Referring now back to FIG. 17, table 1702 presents the sequential datasets used in the experiment.

Inventors used 16S rRNA gene sequencing to distinguish between pathological and control cases, such as Inflammatory bowel disease (IBD), Crohn's disease (CD), Cirrhosis, and different food allergies (milk, nut, and peanut), as well as between 167 subgroups of healthy populations by variables such as ethnicity and sex.

Inventors preprocessed the samples via the MIPMLP pipeline [46]. Inventors merged the features of the species taxonomy using the Sub-PCA method that performs a PCA (Principal component analysis) projection on each group of microbes in the same branch of the cladogram (see Methods). Log normalization was used for the inputs of all the models. When species classification was unknown, Inventors used the best-known taxonomy. Obviously, no information about the predicted phenotype was used during preprocessing.

Before comparing with state-of-the-art methods, Inventors tested 3 baseline models. One was an ASV frequency-based naive model using a two-layer, fully connected neural network. Inventors then compared to the previous state-of-the-art using structure, PopPhy [31], followed by one or two convolutional layers.

Inventors trained all the models on the same datasets, and optimized hyperparameters for the baseline models using an NNI (Neural Network Intelligence) [47] framework on 10 CVs cross validations) of the internal validation set. Inventors measured the models' performance by their Area Under the Receiver Operator Curve (AUC). The best hyperparameters of the ML models used in embodiments described herein were optimized using the precise same setting.

To show that the combination of the ASVs counts of each taxon through the cladogram is useful, Inventors first propose gMic. Inventors created a cladogram for each dataset whose leaves are the preprocessed observed samples (each at its appropriate taxonomic level), as described herein. The internal vertices of the cladogram were populated with the average over their direct descendants at a finer level (e.g., for the family level, Inventors averaged over all genera belonging to the same family). The tree was represented as a graph. This graph was used as the convolution kernel of a GCN, followed by two fully-connected layers to predict the class of the sample. Inventors denote the resulting approach gMic. Inventors used two versions of gMic: In the simpler version, Inventors ignored the microbial count and the frequencies of all existing taxa were replaced by a value of 1, and only the cladogram structure was used. In the second version, gMic+v, Inventors used the normalized taxa frequency values as the input.

Inventors compared the AUC on a 10 CVs test set of gMic and gMic+v to the state-of-the-art results on the same datasets. The AUC, when using only the structure in gMic was similar to one of the best naive models using the ASVs' frequencies as tabular data. When combined with the ASVs' frequencies, gMic+v outperformed existing methods in 4 out of 9 datasets by 0.05 on average.

Referring now back to FIG. 18, graph 1802 presents the comparison between model performance performed as part of the Experiment described herein. The average AUC is measured on the external test set on 9 different phenotypes. Each subplot is a phenotype. The stars represent the significance of the p-value (after Benjamini Hochberg correction) on the external test set. If there were differences in the significance on the 10 CVs and the external test set, the different corrected p-value of the 10 CVs is reported in brackets, *−p≤0.05, **−p≤0.01, ***−p≤0.001. For each box, the three rightmost set of plots denote the baseline. The two most right most bars denote the current best baseline. The third bar from the right denote the best baseline obtained using the MIPMLP. The three central bars denote the iMic AUC using either a one or two-dimensional CNN. The three leftmost bars denote gMic (either gMic or gMic+V). Inventors also added the iMic results to allow for a comparison.

Referring now back to FIG. 19, table 1902 presents 10 CVs mean performances with standard deviation (std) on external test sets. The std is the std among CV folds.

While gMic captures the relation between similar taxa, it still does not solve the sparsity problem. Inventors thus suggest using iMic for a different combination of the relation between the structure of the cladogram and the taxa's frequencies into an image and applying CNNs on this image to classify the samples.

iMic is initiated with the same tree as gMic, but then instead of a GCN, the cladogram with the means in the vertices is projected to a two-dimensional matrix with 8 rows (the number of taxonomic levels in the cladogram), and a column for each leaf.

Each leaf is set to its preprocessed frequency at the appropriate level and zero at the finer level (so if an ASV is at the genus level, the species level of the same genus is 0). Values at a coarser level (say at the family level) are the average values of the level below (a finer level-genus). For example, if there are 3 genera belonging to the same family, at the family level, the 3 columns will receive the average of the 3 values at the genus level.

As a further step to include the taxonomy in the image, columns were sorted recursively, so that taxa with more similar frequencies in the dataset would be closer using hierarchical clustering on the frequencies within a subgroup of taxa. For example, assume 3 sister taxa, taxona, taxonb, and taxonc, the order of those 3 taxa in the row is determined by their proximity in the Dendrogram based on their frequencies.

The test AUC of iMic was significantly higher than the state-of-the-art models in 6 out of 9 datasets by an average increase in AUC of 0.122, as shown in FIG. 18 and/or FIG. 19). Specifically, in all the datasets that were tested, iMic had a significantly higher AUC than the 2 PopPhy models by 0.134 on average (corrected p-value<0.001 of two-sided T-test). Similar results were obtained on shotgun metagenomics datasets. iMic can also be applied to several different cohorts together. Inventors used 4 different IBD cohorts, referred as IBD1 [48], IBD2 [49], IBD3 [50], IBD4 [51]. Three of the cohorts were downloaded from [52]. Some of the datasets had no information at the species taxonomic level. Therefore, all the iMic images were created by the genus taxonomic level. Two learning tasks were applied: The first was a Leave One Dataset Out (LODO) task, where iMic was trained on 3 mixed cohorts and one cohort was left for testing. The second task was mixed learning based on the 4 cohorts. The LODO approach slightly reduced iMic's accuracy, but still had high accuracy (AUC of 0.745 for the LODO IBD1, 0.659 for the LODO IBD2, 0.7 for the LODO IBD3, and 0.63 for the LODO IBD4). Inventors also tested a mixed-learning setup where all datasets were combined with a higher AUC of 0.82+/−0.01.

Often, non-microbial features are available beyond the microbiome. Those can be added to iMic by concatenating the non-microbial features to the flattened microbial output of the last CNN layer before the fully connected (FCN) layers. Adding non-microbial features even further improves the results of iMic when compared to a model without non-microbial features. Moreover, the incorporation of non-microbial features (such as sex, HDM, atopic dermatitis, asthma, age, and dose of allergen in the Allergy learning) leads to a higher accuracy than their incorporation in standard models, for example, FIG. 8.

As discussed herein, microbiome-based ML is hindered by multiple technical challenges, including for example, several representation levels, high sparsity, and high dimensional input vs a small number of samples. iMic solves these technical challenges, by simultaneously using (e.g., all) known taxonomic levels. Moreover, iMic resolves sparsity by ensuring ASVs with similar taxonomy are nearby and averaged at finer taxonomic levels. As such, even if each sample has different ASVs, there is still common information at finer taxonomic levels. Using perturbations on the original samples, Inventors demonstrate that iMic copes with each of these challenges better than existing methods. For example:
High sparsity. The microbiome data is extremely sparse with most of the taxa absent from most of the samples. iMic CNN averages over neighboring taxa. As such, even in the absence of some taxa, it can still infer their expected value from their neighbors. Inventors define the initial sparsity rate of the data as the fraction of zero entries from the raw ASVs. The least sparse data was the Cirrhosis dataset (72%), followed by IBD, CD, CA, Ravel, and MF (all with a sparsity of 96%). The sparsest dataset was Allergy (98%) which was not used in the sparsity analysis. Inventors randomly zeroed entries to reach the required simulated sparsities (75, 80, 85, 90, and 95% for the Cirrhosis dataset) and (97, 98, and 99% for the others). iMic had the highest AUC and the least decrease in AUC when compared to other models (e.g., see FIG. 20, graphs 2004, 2006, and 2008). iMic is also significantly (after Benjamini Hochberg correction) more stable than gMic+v (p-value<0.05). The results are similar for the other datasets. The differences between iMic and all the other models in the AUC and in the decrease in AUC are also significant (after Benjamini Hochberg correction) in CD, CA, Cirrhosis and MF (p-value<0.05).
High dimensional input vs a small number of samples. By using CNNs with strides on the image microbial input, iMic reduces the model's number of parameters in comparison to FCNs. iMic's stability when changing the size of the training set was measured by reducing the training set size and measuring the AUC of iMic as well as the naive models (RF, SVC, LR and FCN). iMic was significantly (after Benjamini Hochberg correction) the most stable model (p-value<0.05) in CA, CD, Cirrhosis, M F, and Allergy, among the models that succeeded to learn (baseline AUC>0.55) as measured by the difference between the AUC of the reduced model and the baseline model, see FIG. 20, graphs 2010, 2012, and 2014.

Referring now back to FIG. 20, graphs include: 2004: Average test AUC (over 10 CVs) as a function of the different sparsity levels, where the first point is the AUC of the original sparsity level (72%, “baseline”) on the Cirrhosis dataset. iMic has the highest AUCs for all simulated sparsity levels (purple line). The error bars represent the standard errors. 2006: Average change in AUC (AUC—baseline AUC) as a function of the sparsity level on the Cirrhosis dataset. 2008: Overall average in AUC change in all the other datasets apart from Cirrhosis. 2010: Average AUC as a function of the number of samples in the training set (Cirrhosis dataset). The error bars represent the standard errors of each model over the 10 CVs. 2012: Average change in AUC (AUC—baseline AUC) as a function of the percent of samples in the training set. 2014: Overall average AUC change over all the algorithms that managed to learn (baseline AUC>0.55) as a function of the percent of samples in the training set.
Several representation levels. iMic uses all taxonomic levels by adding the structure of the cladogram and translating it into an image. iMic further finds the best representation as an image by reordering the columns of the rows using for example, dendrogram clustering while maintaining the taxonomy structure. Inventors confirmed that the reordering of sister taxa according to their similarity improves the performance in the classification task. The average AUCs of all the datasets are significantly higher (after Benjamini Hochberg correction) with taxa ordering vs no ordering (p-value<0.001).

Referring now back to FIG. 21, graphs 2102 indicate the importance of ordering taxa. As seen in graphs 2102, ordering of the tax improves performance of the ML model. The x-axis represents the average AUC over 10 CVs and the y-axis represents the different datasets used. The darker colored bars represent the AUC on the images without taxa reordering, while the lighter colored bars represent the AUC on the images with the Dendrogram reordering with standard errors. All the differences between the AUCs are significant after Benjamini Hochberg correction (p-value<0.001). All the AUCs are calculated on an external test set for each CV. Quite similar results were obtained on the 10 CVs.

Beyond its improved performance, iMic can be used to detect the taxa most associated with a condition. Inventors used Grad-Cam (an explainable AI platform [53]) to estimate the part of the image used by the model to classify each class [54]. Formally, Inventors estimated the gradient information flowing into the first layer of the CNN to assign importance and averaged the importance of the pixels for control and case groups separately (e.g., using the CD dataset, Inventors identify microbes to distinguish patients with CD from healthy subjects). Interestingly, the CNN is most affected by the family and genus level (fifth row and sixth row in images J02 and J04 of FIG. 12). Different taxa for the case and the control (see FIG. 13). To find the microbes that most contributed to the classification, Inventors projected the computed Grad-Cam values back to the cladogram (see FIG. 13). In the CD dataset, Proteobacteria are characteristic of the CD group, in line with the literature. This phylum is proinflammatory and associated with the inflammatory state of CD and overall microbial dysbiosis [55]. Also in line with previous findings is the family Micrococcaceae associated with colonial CD [56] and even with mesenteric adipose tissue microbiome in CD patients [57]. The control group was characterized by the family Bifidobacteriaceace, known for its anti-inflammatory properties, pathogen resistance, and overall improvement of host state [58, 59], and by Akkermansia, which is a popular candidate in the search for next-generation probiotics due to its ability to promote metabolism and the immune function [60].

To test that the significant taxa contribute to the classification, Inventors defined “good columns” and “bad columns”. A “good column” is defined as a column where the sum of the averaged Grad-Cams in the case and control groups is in the top percentile k percentiles, and a “bad column” is defined by the lowest k percentiles. When removing the “good columns”, the test AUC was reduced by 0.07 on average, whereas when the “bad columns” were removed, the AUC slightly improved by 0.006.

Referring now back to FIG. 22, graphs depicting interpretation tests on the CD dataset (2202), the IBD dataset (2204), the Cirrhosis dataset (2206), and the Ravel dataset (2208), are presented. Average AUC values over 10 CVs on the external test set. The x-axis represents the fraction of removed columns. The dark bars represent the performance when all of the columns with Grad-Cams values lower than this fraction have been removed and the light bars represent the performance when the columns with scores above this fraction have been removed. The black line represents the average AUC over 10 CVs of the original model with all the input columns. Results from the other datasets were similar. Removing the top scoring columns always reduced the performance. Removing the bottom scoring columns increases or does not change the AUC.

To ensure that the improved performance is not the result of hyperparameter tuning, Inventors checked the impact on the AUC of fixing all the hyperparameters but one and changing a specific hyperparameter by increasing or decreasing its value by 10-30 percent. The difference between the AUC of the optimal parameters and all the varied combinations is low with a range of 0.03+/−0.03, smaller than the increase in AUC of iMic compared to other methods.

iMic translates the microbiome into an image. One can use the same logic and translate a set of microbes to a movie to classify sequential microbiome samples. Inventors used iMic to produce a 2-dimensional representation for the microbiome of each time step and combined those into a movie of the microbial images. Inventors used a 3D Convolutional Neural Network (3D-CNN) to classify the samples. Inventors applied 3D-iMic to 2 different previously studied temporal microbiome datasets, comparing the results based on embodiments described herein to the state-of-the-art—a one dimensional representation of taxon-NN PhyLoSTM [61]. The AUC of 3D-iMic is significantly higher after Benjamini Hochberg correction (p—value<0.0005) than the AUC of PhyloLSTM over all datasets and tags.

Referring now back to FIG. 23, graphs 2302 comparing performance of 3D learning vs PhyLoSTM based on experimental results are presented. The AUCs of the 3D-iMic are consistently higher than the AUCs of the phyLoSTM on all the tags and datasets Inventors checked (n=5). The standard errors among the CVs are also shown. phyLoSTM is the current state-of-the-art for these datasets (two-sided T-test, p-value<0.0005).

To understand what temporal features of the microbiome were used for the classification, Inventors calculated again the heatmap of backwards gradients of each time step separately using Grad-Cam. Inventors focused on CNNs with a window of 3-time points, and represented the heatmap of the contribution of each pixel in each time step in the R, G, and B channels, producing an image that combines the cladogram and time effects and projected this image on the cladogram. Inventors used this visualization on the DiGiulio case-control study of preterm and full-term neonates' microbes, and again projected the microbiome on the cladogram, showing the RGB representation of the contribution to the classification. Again, characteristic taxa of preterm infants (Image 1402 of FIG. 14 and cladogram 1502 of FIG. 15) and full-term infants (Image 1404 of FIG. 14 and cladogram 1504 of FIG. 15) were in line with previous research. Here preterm infants were characterized by TM7, common in the vaginal microbiota of women who deliver preterm ([3, 62]). Staphylococci have also been identified as the main colonizers of the pre-term gut ([63, 64]). Full-term infants were characterized by a number of Fusobacteria taxa. Bacteria of this phylum are common at this stage of life [65].

The application of ML to microbial frequencies, represented by 16S rRNA or shotgun metagenomics ASV counts at a specific taxonomic level is affected by 3 types of information loss—ignoring the taxonomic relationships between taxa, ignoring sparse taxa present in only a few samples, and ignoring rare taxa (taxa with low frequencies) in general.

Inventors have first shown that the cladogram is highly informative through a graph-based approach named gMic. Inventors have shown that even completely ignoring the frequency of the different taxa, and only using their absence or presence can lead to highly accurate predictions on multiple ML tasks, typically as good or even better than the current state-of-the-art.

Inventors then propose an image-based approach named iMic to translate the microbiome to an image where similar taxa or proximal are close to each other and apply CNN to such images to perform ML tasks. Inventors have shown that iMic produces higher precision predictions (as measured by the test set shown in FIG. 18) than current state-of-the-art microbiome-based ML on a wide variety of ML tasks. Inventors then have further shown that iMic is less sensitive to the limitations above. Specifically, iMic is less sensitive to the rarefaction of the ASV in each sample. Removing random taxa from samples had the least effect on iMic's accuracy in comparison to other methods. Similarly, iMic is most robust to the removal of full samples. Finally, iMic explicitly incorporates the cladogram. Removing the cladogram information reduces the classification accuracy. iMic also improves the state-of-the-art in microbial dynamic prediction (phyLoSTM) by treating the dynamic microbiome as a movie and applying 3D-CNNs. Inventors found that a typical window of 3 snapshots was enough to extract the information from dynamic microbiome samples.

An important advantage of iMic is the production of explainable models. Moreover, treating the microbiome as images opens the door to many vision-based ML tools, such as: transfer learning from pre-trained models on images, self-supervised learning, and data augmentation. Combining iMic with an explainable AI methodology highlights microbial taxa associated as a group with different phenotypes. Those are in line with relevant taxa previously noted in the literature.

While iMic handles many limitations of existing methods, it still has important limitations and arbitrary decisions. iMic orders taxa by hierarchically using the cladogram, and within the cladogram, based on the similarity between the counts among neighboring microbes. This is only one possible clustering method and other orders may be used that may further improve the accuracy. Also, Inventors used a simple network structure; however, much more complex structures could be used. Still iMic shows that the detailed incorporation of the structure is crucial for microbiome-based ML.

Other limitations of iMic include: A) While iMic improves ML, it does not produce a distance metric, and such distance metric may be developed. B) iMic learns on the full dataset and does not directly define specific single microbes linked to the outcome. This is addressed by applying explainable AI methods (specifically Grad-Cam) to the iMic results. C) As is the case for any ML, it does not provide causality. Still composite biomarkers, based on a full microbiome repertoire are possible.

The development of microbiome-based biomarkers (micmarkers) is one of the most promising routes for easy and large-scale detection and prediction. However, while many microbiome based prediction algorithms have been developed, they suffer from multiple limitations, which are mainly the result of the sparsity and the skewed distribution of taxa in each host. iMic and gMic are important steps in the translation of microbiome samples from a list of single taxa to a more holistic view of the full microbiome. Inventors are now developing multiple microbiome based diagnostics, including a prediction of the effect of the microbiome composition on Fecal Microbiota Transplants (FMT outcomes)[66]. Inventors have previously shown that the full microbiome (and not specific microbes) can be used to predict pregnancy complications [67]. Inventors propose that either the tools developed here or tools using the same principles can be used for high-accuracy clinical microbiome-based biomarkers.

### Methods

### Preprocessing

Inventors preprocessed the 16S rRNA gene sequences of each dataset using the MIPMLP pipeline [46]. The preprocessing of MIPMLP contains 4 stages: merging similar features based on the taxonomy, scaling the distribution, standardization to z-scores, and dimension reduction. Inventors merged the features at the species taxonomy by Sub-PCA before using all the models. Inventors performed log normalization as well as z-scoring on the patients. No dimension reduction was used at this stage. For the LODO and mixed predictions of the IBD datasets, the features were merged into the genus taxonomic level, since the species of 3 of the cohorts were not available.

### All Models Preprocessing

Sub-PCA merging in MIPMLP. A taxonomic level (e.g., species) is set. All the ASVs consistent with this taxonomy are grouped. A PCA is performed on this group. The components which explain more than half of the variance are added to the new input table. This was applied for all models apart from the PopPhy models [31].

Log normalization in MIPMLP. Inventors logged (10 base) scale the features element-wise, according to the following formula:

$${x}_{i , j} \to log ( {x}_{i , j} + ϵ ) \qquad Equation ( 1 )$$

where ϵ is a minimal value (=0.1) to prevent log of zero values. This was applied for all models apart from the PopPhy models.

### PopPhy Preprocessing

Sum merging in MIPMLP. A level of taxonomy (e.g., species) is set. All the ASVS consistent with this taxonomy are grouped by summing them. This was applied to the PopPhy models.

Relative normalization in MIPMLP. To normalize each taxon through its relative frequency:

$${x}_{i , j} = \frac{{x}_{i , j}}{{\sum }_{k = 1}^{n} {x}_{k , j}} \qquad Equation ( 2 )$$

Inventors normalized the relative abundance of each taxon j in sample i by its relative abundance across all n samples. This was applied for the PopPhy models.

### Baseline Algorithms

We compared gMic and iMic models' results to 6 baseline models: ASV frequency two layers fully connected neural network (FCN). The FCN was implemented via the pytorch lightening platform [68]. Other simple popular value-based approaches are: Random Forest (RF) [69], Support Vector Classification (SVC) [69] and Logistic Regression (LR) [69]. All the simple approaches were implemented by the sklearn functions: sklearn.ensemble.RandomForestClassifier, sklearn.svm.SVC and sklearn.linear model.LogisticRegression, respectively. To evaluate the performance by using only the values. The other two models were the previous state-of-the-art models that use structure, PopPhy [31], followed by 1 convolutional layer or 2 convolutional layers and their output followed by FCNs (2 layers). The models' inputs were the ASVs merged at the species level by the sum method followed by a relative normalization as described. Inventors used the original PopPhy code from Reiman's GitHub.

iMic

The iMic's framework includes 3 processes: Populating the mean cladogram, Cladogram2Matrix, and feed into a ML model (e.g., CNN).

Given a vector of log-normalized ASVs frequencies merged to taxonomy 7-b, each entry of the vector, bi represents a microbe at a certain taxonomic level. Inventors built an average cladogram, where each internal node is the average of its direct children. Once the cladogram was populated, Inventors built the representation matrix.

Inventors created a matrix R ∈R8×N, where N was the number of leaves in the cladogram and 8 represents the 8 taxonomic levels, such that each row represents a taxonomic level. Inventors added the values layer by layer, starting with the values of the leaves. If there were taxonomic levels below the leaf in the image, they were populated with zeros. Above the leaves, Inventors computed for each taxonomic level the average of the values in the layer below. If the layer below had k different values, Inventors set the average to all k positions in the current layer. For example, if there were 3 species within one genus with values of 1,3 and 3. Inventors set a value of 7/3 to the 3 positions at the genus level including these species. Inventors reordered the microbes at each taxonomic level (row) to ensure that similar microbes are close to each other in the produced image. Specifically, Inventors built a Dendrogram based on the Euclidean distances as a metric using complete linkage on the columns, relocating the microbes according to the new order while keeping the phylogenetic structure. The order of the microbes was created recursively. Inventors started by reordering the microbes on the phylum level, relocating the phylum values with all their sub-tree values in the matrix. Then Inventors built a Dendrogram of the descendants of each phylum separately, reordering them and their sub-tree in the matrix. Inventors repeated the reordering recursively until all the microbes in the species taxonomy of each phylum were ordered.

### 2-Dimensional CNN

The microbiome matrix was used as the input to a standard CNN [70]. Inventors tested both one and two convolution layers (when 3 convolution layers or more were used, the models suffered from over-fitting). The loss function was the binary cross entropy. Inventors used L1 regularization. Inventors also used a dropout after each layer, the strength of the dropout was controlled by a hyperparameter. For each dataset, Inventors chose the best activation function among RelU, elU, and tanh. Inventors also used strides and padding. Examples of the hyperparameter ranges as well as the chosen and fixed hyperparameters are described herein. In order to limit the number of model parameters, Inventors 462 added max pooling between the layers if the number of parameters was higher than 5000. The output of the CNNs was the input of a two-layer, fully connected neural network.

gMic and gMic+v

The cladogram and the gene frequency vector were used as the input. The graph was represented by the symmetric normalized adjacency matrix, which was denoted A^{˜} as can be seen in the following equations:

$$\tilde{A} = {D}^{- \frac{1}{2}} {AD}^{- \frac{1}{2}} \qquad Equation ( 3 )$$

$$σ ( ( \tilde{A} + α \cdot I ) \cdot sign ( υ ) \cdot W ) FCN \qquad Equation ( 4 )$$

The loss function was binary cross entropy. In this model, Inventors used L2 regularization as well as a dropout.

gMic

The cladogram was built and populated as in iMic. In gMic, a GCN layer was applied to the cladogram. The output of the GCN layer was the input to a fully connected neural network (FCN) as in:

$$σ ( ( \tilde{A} + α \cdot I ) \cdot sign ( υ ) \cdot W ) FCN \qquad Equation ( 5 )$$

where v denotes the ASVs frequency vector (all the cladogram's vertices, and not only the leaves, in contrast with b above), sign(v) denotes the same vector where all positive values were replaced by 1 in the gene frequency vector (i.e., the values are ignored). α denotes a learned parameter, regulating the importance given to the vertices' values against the first neighbors, W denotes the weight matrix in the neural network, and σ denotes the activation function. The architecture of the FCN is common in all datasets. The hyperparameters may be different: two hidden layers, each followed by an activation function.

### Data

Inventors used 9 different tags from 6 different datasets of 16S rRNA ASVs to evaluate iMic and gMic+v. 4 datasets were contained within the Knights Lab ML repository [71]: Cirrhosis, Caucasians and Afro Americans (CA), Male vs Female (MF) and Ravel vagina.
The Cirrhosis dataset was taken from a study of 68 Cirrhosis patients and 62 healthy subjects [72].
The MF dataset was a part of the human microbiome project (HMP) and contained 98 males and 82 females. [73].
The CA dataset consisted of 104 Caucasian and 96 Afro-American vaginal samples [74].
The Ravel dataset was based on the same cohort as the CA, but checked another condition of the Nugent score [74]. The Nugent score is a Gram stain scoring system for vaginal swabs to diagnose bacterial vaginosis.
The IBD dataset contains 137 samples with inflammatory bowel disease (IBD), including Crohn's disease (CD) and ulcerative colitis (UC), and 120 healthy samples as controls. Inventors also used the same dataset for another task of predicting only CD from the whole society, where there were 94 with CD and 163 without CD [48].
The Allergy dataset is a cohort of 274 subjects. Inventors tried to predict 3 different outcomes. The first is having or not having a milk allergy, where there are 74 subjects with a milk allergy and 200 without. The second is having or not having a nut (walnut and hazelnut) allergy, where there are 53 with a nut allergy and 221 without. The third is having or not having an allergy to peanut, where 79 have a peanut allergy and 195 do not [5].

For the comparisons with TopoPhy, Inventors applied iMic to the shotgun metagenomes datasets presented in TopoPhy [33] from MetAML, including a Cirrhosis dataset with 114 cirrhotic patients and 118 healthy subjects (“Cirrhosis-2”), an obesity dataset with 164 obese and 89 non-obese subjects (“BMI”), a T2D dataset of 170 T2D patients and 174 control samples (“T2D”).

For the comparisons with TaxoNN, Inventors applied iMic to the datasets presented in TaxoNN's paper (Cirrhosis-2 and T2D). For the comparison with DeepEn-Phy [34], Inventors applied iMic on the Guangdong Gut Microbiome Project (GGMP)—a large microbiome-profiling study conducted in Guangdong Province, China, with 7009 stool samples (2269 cases and 4740 controls to classify smoking status). GGMP was downloaded from the Qiita platform. Inventors used the results supplied by TopoPhy, TaxoNN, and DeepEn-Phy, and did not apply them to other datasets, since their codes were either missing, or did not work as is, and Inventors did not want to make assumptions regarding the corrections required for the code. Inventors also used two sequential datasets to evaluate iMic-CNN3.
The first dataset was the DIABIMMUNE three-country cohort with food allergy outcomes (Milk, Egg, Peanut, and Overall). This cohort contained 203 subjects with 7.1428 time steps on average. [75].
The second dataset was a DiGiulio case-control study. This was a case-control study comprised of 40 pregnant women, 11 of whom delivered preterm serving as the outcome. Overall, in this study, there were 3767 samples with 1420 microbial samples from 4 body sites: vagina, distal gut, saliva, and tooth/gum. In addition to bacterial taxonomic composition, clinical and demographic attributes included in the dataset were gestational or postpartum day when the sample was collected, race, and ethnicity [76].

### Statistics—Comparison Between Models

To compare the performances of the different models, Inventors performed a one-way ANOVA test (from scipy.stats in python) on the test AUC from the 10 CVs of all the models. If the ANOVA test was significant, Inventors also perform a two-sided T-test between iMic and the other models and between the two CNNs on the iMic representation. Correction for multiple testing (Benjamini-Hochberg procedure, Q) was applied when appropriate with a significance level of Q<0.05. (see FIG. 18). Only significant results after a correction were reported.

To compare the performance on the sparsity and high dimensions challenges, Inventors first performed a two-way ANOVA with the first variable being the sparsity and the second variable being the model on the test AUC over 10 CVs. Only when the ANOVA test was significant (all the datasets in our case), Inventors also performed a two-sided T-test between iMic and the naive models. Correction for multiple testing (Benjamini-Hochberg procedure, Q) was applied when appropriate with significance 541 defined at Q<0.05. All the tests were also checked on the independent 10 CVs of the models on the validation set and the results were similar. Note that in contrast with the test set estimates, this test may be affected by parameter tuning.

### Experimental Setup

### Splitting Data to Training, Validation and Test Sets

Following the initial preprocessing, Inventors divided the data using an external stratified test, such that the distribution of positives and negatives in the training set and the held-out test set would be the same and would preserve the patient identity in cases and controls into training, test, and validation sets. This ensures that the same patient cannot be simultaneously in the training and the test set. The external test was always the same 20 percent of the whole data. The remaining 80 percent were divided into the internal validation (20 percent of the data) and the training set (60 percent). In cross-validations, Inventors changed the training and validations, but not the test.

### Hyperparameters Tuning

Inventors computed the best hyperparameters for each model using a 10-fold CV [77] on the internal validation. Inventors chose the hyperparameters according to the average AUC on the 10 validations. The platform Inventors used for the optimization of the hyperparameters is NNI (Neural Network Intelligence) [47]. The hyperparameters tuned were: the coefficient of the L1 loss, the weight decay (L2-regularization), the activation function (RelU, elU or tanh, which makes the model nonlinear), the number of neurons in the fully connected layers, dropout (a regularization method which zeros the neurons in the layers in the dropout's probability), batch size and learning rate. For the CNN models, Inventors also included the kernel sizes as well as the strides and the padding as hyperparameters. The search spaces Inventors used for each hyperparameter were: L1 coefficient was chosen uniformly from [0,1]. Weight decay was chosen uniformly from [0,0.5]. The learning rate was one of [0.001,0.01,0.05]. The batch size was [32, 64,218,256]. The dropout was chosen universally from [0,0.05,0.1,0.2,0.3,0.4,0.5]. Inventors chose the best activation function from RelU, ElU and tanh. The number of neurons was proportional to the input dimension. The first linear division factor from the input size was chosen randomly from [1,11]. The second layer division factor was chosen from [1,6]. The kernel sizes were defined by two different hyperparameters, a parameter for its length and its width. The length was in the range of [1,8] and the width was in the range of [1,20]. The strides were in the range of [1,9] and the channels were in the range of [1,16]. For the classical ML models, Inventors used a grid search instead of the NNI platform. The evaluation method was similar to the other models. The hyperparameters of the RF were: The number of trees in the range of [10,50,100,150,200] and the function to measure the quality of a split (one of “gini”, “entropy”, “log loss”). The hyperparameters of the SVC were: the regularization parameter in the range of [0.0,0.1,0.2,0.3,0.4,0.5,0.6,0.7,0.8,0.9,1.0], and the kernel (one of “linear”, “poly”, “rbf”, “sigmoid”).

### ML Nomenclature

In order to facilitate the understanding of the more ML oriented terms in the text, a short not necessarily binding description of the main ML terms used in the manuscript is provided. The descriptions are examples and not necessarily limiting.
Model may refer to the mathematical relation between any input (microbiome ASVs in the present disclosure) and the appropriate output (e.g., the sample and/or the phenotype). In ML, the model may include a set of parameters called weights, and the ML trains the model by finding the weights that for which the model is in best agreement with the relation between the input and output in the “Training set”.
Training set may refer to the part of the data used to train the model. The quality of the fit between the input and output data on the training set is necessarily not a good measure of the quality of the model, since it may be an “overfit”.
Overfitting may refer to a problem occurring when a model produces good results on data in the training set (usually due to too many parameters), but produces poor results on unseen data.
Validation set may refer to a separate set from the training set that is used to monitor, but is not used for the training process. This set can be used to optimize some parts of the learning process including setting the “hyperparameters”.
Model hyperparameters may refer to adjustable values that are not considered part of the model itself in that they are not updated during training, but which still have an impact on the training of the model and its performance. To ensure that those are not fitted to maximize the test set performances, the hyperparameters are optimized using an internal validation set.
Test set may refer to data used to test the model that is not used for either hyperparameter optimization or the training. The quality estimated on the test set is the most accurate estimate of the accuracy.
10-Fold Cross-Validation (referred to as 10 CVs) may refer to a resampling procedure used to evaluate machine learning models on a limited data sample. The data may be first partitioned into for example, 10 equally (or nearly equally or other value) sized segments or folds. Subsequently, 10 iterations (or other number) of training and validation may be performed such that within each iteration a different fold of the data is held-out for validation while the remaining 9 folds are used for training.
Receiver Operating Characteristic Curve (ROC) may refer to a graph showing the performance of a classification model at all classification thresholds. This curve plots two parameters: True Positive Rate (TPR=is the probability that an actual positive will test positive). False Positive Rate (FPR=the probability that an actual negative will test positive).
Area under the ROC curve (AUC) may refer to a single scalar value that measures the overall performance of a binary classifier. The AUC value is within the range [0.5-1.0], where the minimum value represents the performance of a random classifier and the maximum value would correspond to a perfect classifier (e.g., with a classification error rate equivalent to zero). It measures the area under the ROC curve we define above.

### REFERENCES

[1] Supinda Bunyavanich, Nan Shen, Alexander Grishin, Robert Wood, Wesley Burks, Peter Dawson, Stacie M. Jones, Donald Y. M. Leung, Hugh Sampson, Scott Sicherer, et al. Early life gut microbiome composition and milk allergy resolution. Journal of Allergy and Clinical Immunology, 138(4):1122-1130, 2016.
[2] Khui Hung Lee, Jing Guo, Yong Song, Amir Ariff, Michael O'sullivan, Belinda Hales, Benjamin J. Mullins, and Guicheng Zhang. Dysfunctional gut microbiome networks in childhood IgE-mediated food allergy. International Journal of Molecular Sciences, 22(4):2079, 2021.
[3] Jennifer M. Fettweis, Myrna G. Serrano, J. Paul Brooks, David J. Edwards, Philippe H. Girerd, Hardik I. Parikh, Bernice Huang, Tom J. Arodz, Laahirie Edupuganti, Abigail L. Glascock, et al. The vaginal microbiome and preterm birth. Nature mMedicine, 25(6):1012-1021, 2019.
[4] Sonia Michail, Matthew Durbin, Dan Turner, Anne M. Griffiths, David R. Mack, Jeffrey Hyams, Neal Leleiko, Harshavardhan Kenche, Adrienne Stolfi, and Eytan Wine. Alterations in the gut microbiome of children with severe ulcerative colitis. Inflammatory Bowel Diseases, 18(10):1799-1808, 2012.
[5] Michael R. Goldberg, Hadar Mor, Dafna Magid Neriya, Faiga Magzal, Efrat Muller, Michael Y. Appel, Liat Nachshon, Elhanan Borenstein, Snait Tamir, Yoram Louzoun, et al. Microbial signature in IgE-mediated food allergies. Genome Medicine, 12(1):1-18, 2020.
[6] Dana Binyamin, Nir Werbner, Meital Nuriel-Ohayon, Atara Uzan, Hadar Mor, Atallah Abbas, Oren Ziv, Raffaele Teperino, Roee Gutman, and Omry Koren. The aging mouse microbiome has obesogenic characteristics. Genome Medicine, 12(1):1-9, 2020.
[7] David M. Ward, RolandWeller, and Mary M. Bateson. 16s rRNA sequences reveal numerous uncultured microorganisms in a natural community. Nature, 345(6270):63-65, 1990.
[8] Rachel Poretsky, Luis M. Rodriguez-R, Chengwei Luo, Despina Tsementzi, and Konstantinos T Konstantinidis. Strengths and limitations of 16s rRNA gene amplicon sequencing in revealing temporal microbial community dynamics. PloS One, 9(4):e93827, 2014.
[9] Alastair B Ross, Stephen J Bruce, Anny Blondel-Lubrano, Sylviane Oguey-Araymon, Maurice Beaumont, Alexandre Bourgeois, Corine Nielsen-Moennoz, Mario Vigo, Laurent-Bernard Fay, Sunil Kochhar, et al. A whole-grain cereal-rich diet increases plasma betaine, and tends to decrease total and ldl-cholesterol compared with a refined-grain diet in healthy subjects. British journal of nutrition, 105(10):1492-1502, 2011.
[10] Vinod K Gupta, Minsuk Kim, Utpal Bakshi, Kevin Y Cunningham, John M Davis I I I, Konstantinos N Lazaridis, Heidi Nelson, Nicholas Chia, and Jaeyun Sung. A predictive index for health status using species-level gut microbiome profiling. Nature communications, 11(1):4635, 2020.
[11] JinfengWang, Jiayong Zheng, Wenyu Shi, Nan Du, Xiaomin Xu, Yanming Zhang, Peifeng Ji, Fengyi Zhang, Zhen Jia, Yeping Wang, et al. Dysbiosis of maternal and neonatal microbiota associated with gestational diabetes mellitus. Gut, 67(9):1614-1625, 2018.
[12] Andrei Prodan, Valentina Tremaroli, Harald Brolin, Aeilko H. Zwinderman, Max Nieuwdorp, and Evgeni Levin. Comparing bioinformatic pipelines for microbial 16s rRNA amplicon sequencing. PLoS One, 15(1):e0227434, 2020.
[13] John L. Darcy, 634 Alex D. Washburne, Michael S. Robeson, Tiffany Prest, Steven K. Schmidt, and Catherine A. Lozupone. A phylogenetic model for the recruitment of species into microbial communities and application to studies of the human microbiome. The ISME Journal, 14(6):1359-1368, 2020.
[14] Yu-Qing Qiu, Xue Tian, and Shihua Zhang. Infer metagenomic abundance and reveal homologous genomes based on the structure of taxonomy tree. IEEE/ACM Transactions on Computational Biology and Bioinformatics, 12(5):1112-1122, 2015.
[15] Ehsaneddin Asgari, Philipp C Mu'nch, Till R. Lesker, Alice C. McHardy, and Mohammad R. K. Mofrad. Ditaxa: nucleotide-pair encoding of 16s rrna for host phenotype and biomarker detection. Bioinformatics, 35(14):2498-2500, 2019.
[16] Huisong Lee, Hyeon Kook Lee, Seog Ki Min, and Won Hee Lee. 16s rDNA microbiome composition pattern analysis as a diagnostic biomarker for biliary tract cancer. World Journal of Surgical Oncology, 18(1):1-10, 2020.
[17] Sondra Turjeman and Omry Koren. Using the microbiome in clinical practice, 2021.
[18] Suguru Nishijima, Wataru Suda, Kenshiro Oshima, Seok-Won Kim, Yuu Hirose, Hidetoshi Morita, and Masahira Hattori. The gut microbiome of healthy Japanese and its microbial and functional uniqueness. DNA Research, 23(2):125-133, 2016.
[19] Random forest algorithm for predicting chronic diabetes disease. Special issue on Advancements in Applications of Microbiology and Bioinformatics in Pharmacology, pages 4-8, 2020.
[20] Jonathan P. Jacobs, Maryam Goudarzi, Namita Singh, Maomeng Tong, Ian H. McHardy, Paul Ruegger, Miro Asadourian, Bo-Hyun Moon, Allyson Ayson, James Borneman, et al. A disease-associated microbial and metabolomics state in relatives of pediatric inflammatory bowel disease patients. Cellular and Molecular Gastroenterology and Hepatology, 2(6):750-766, 2016.
[21] Meirav Ben Izhak, Adi Eshel, Ruti Cohen, Liora Madar-Shapiro, Hamutal Meiri, Chaim Wachtel, Conrad Leung, Edward Messick, Narisra Jongkam, Eli Mayor, et al. Projection of gut microbiome pre- and post-bariatric surgery to predict surgery outcome. mSystems, 6(3):e01367-20, 2020.
[22] Mai Oudah and Andreas Henschel. Taxonomy-aware feature engineering for microbiome classification. BMC Bioinformatics, 19(1):1-13, 2018.
[23] Davide Albanese, Carlotta De Filippo, Duccio Cavalieri, and Claudio Donati. Explaining diversity in metagenomic datasets by phylogenetic-based feature weighting. PLoS Computational Biology, 11(3):e1004186, 2015.
[24] Jian Xiao, Li Chen, Yue Yu, Xianyang Zhang, and Jun Chen. A phylogeny-regularized sparse regression model for predictive modeling of microbial community data. Frontiers in Microbiology, 9:3112, 2018.
[25] Gregory Ditzler, Robi Polikar, and Gail Rosen. Multi-layer and recursive neural networks for metagenomic classification. IEEE Transactions on Nanobioscience, 14(6):608-616, 2015.
[26] Alex Krizhevsky, Ilya Sutskever, and Geoffrey E. Hinton. Imagenet classification with deep convolutional neural networks. Advances in Neural Information Processing Systems, 674 25:1097-1105, 2012.
[27] Thomas N. Kipf and Max Welling. Semi-supervised classification with graph convolutional networks. arXiv preprint arXiv:1609.02907, 2016.
[28] Eunbyung Park, Xufeng Han, Tamara L. Berg, and Alexander C. Berg. Combining multiple sources of knowledge in deep CNNs for action recognition. In 2016 IEEE Winter Conference on Applications of Computer Vision (WACV), pages 1-8. IEEE, 2016.
[29] Jinfeng Bai, Zhineng Chen, Bailan Feng, and Bo Xu. Image character recognition using deep convolutional neural network learned from different languages. In 2014 IEEE International Conference on Image Processing (ICIP), pages 2560-2564. IEEE, 2014.
[30] Wenging Sun, Bin Zheng, and Wei Qian. Computer aided lung cancer diagnosis with deep learning algorithms. In Medical Imaging 2016: Computer-Aided Diagnosis, volume 9785, page 97850Z. International Society for Optics and Photonics, 2016.
[31] Derek Reiman, Ahmed A. Metwally, Jun Sun, and Yang Dai. PopPhy-CNN: a phylogenetic tree embedded architecture for convolutional neural networks to predict host phenotype from metagenomic data. IEEE Journal of Biomedical and Health Informatics, 24(10):2993-3001, 2020.
[32] Divya Sharma, Andrew D. Paterson, and Wei Xu. TaxoNN: ensemble of neural networks on stratified microbiome data for disease prediction. Bioinformatics, 36(17):4544-4550, 2020.
[33] Bojing Li, Duo Zhong, Xingpeng Jiang, and Tingting He. TopoPhy-CNN: Integrating topological information of phylogenetic tree for host phenotype prediction from metagenomic data. In 2021 IEEE International Conference on Bioinformatics and Biomedicine (BIBM), pages 456-461. IEEE, 2021.
[34] Wodan Ling, Youran Qi, Xing Hua, and Michael C Wu. Deep ensemble learning over the microbial phylogenetic tree (DeepEn-Phy). In 2021 IEEE International Conference on Bioinformatics and Biomedicine (BIBM), pages 470-477. IEEE, 2021.
[35] Elliott Gordon-Rodriguez, Thomas P Quinn, and John P Cunningham. Learning sparse log-ratios for high-throughput sequencing data. Bioinformatics, 38(1):157-163, 2022.
[36] Saad Khan and Libusha Kelly. Multiclass disease classification from microbial whole community metagenomes. In Pacific Symposium on Biocomputing 2020, pages 55-66. World Scientific, 2019.
[37] Yu Guang Wang, Ming Li, Zheng Ma, Guido Montufar, Xiaosheng Zhuang, and Yanan Fan. Haar graph pooling. Proceedings of the 37th International Conference on Machine Learning, 2020.
[38] Frederik Diehl. Edge contraction pooling for graph neural networks. arXiv preprint arXiv:1905.10990, 2019.
[39] Enxhell Luzhnica, Ben Day, and Pietro Lio. Clique pooling for graph classification. RLGM, ICLR 2019, 2019.
[40] Rex Ying, Jiaxuan You, Christopher Morris, Xiang Ren, William L. Hamilton, and Jure Leskovec. Hierarchical graph representation learning with differentiable pooling. arXiv preprint arXiv:1806.08804, 2018.
[41] Hao Yuan and Shuiwang Ji. Structpool: Structured graph pooling via conditional random fields. In Proceedings of the 8th International Conference on Learning Representations, 2020.
[42] Yao 716 Ma, SuhangWang, Charu C. Aggarwal, and Jiliang Tang. Graph convolutional networks with eigenpooling. In Proceedings of the 25th ACM SIGKDD International Conference on Knowledge Discovery & Data Mining, pages 723-731, 2019.
[43] Omer Nagar, Shoval Frydman, Ori Hochman, and Yoram Louzoun. Quadratic gcn for graph classification. arXiv preprint arXiv:2104.06750, 2021.
[44] Quy Cao, Xinxin Sun, Karun Rajesh, Naga Chalasani, Kayla Gelow, Barry Katz, Vijay H. Shah, Arun J. Sanyal, and Ekaterina Smirnova. Effects of rare microbiome taxa filtering on statistical analysis. Frontiers in Microbiology, 11:607325, 2021.
[45] Chao Xiong, Ji-Zheng He, Brajesh K Singh, Yong-Guan Zhu, Jun-Tao Wang, Pei-Pei Li, Qin-Bing Zhang, Li-Li Han, Ju-Pei Shen, An-Hui Ge, et al. Rare taxa maintain the stability of crop mycobiomes and ecosystem functions. Environmental Microbiology, 23(4):1907-1924, 2021.
[46] Yoel Jasner, Anna Belogolovski, Meirav Ben-Itzhak, Omry Koren, and Yoram Louzoun. Microbiome preprocessing machine learning pipeline. Frontiers in Immunology, 12, 2021.
[47] Microsoft. Neural Network Intelligence, 1 2021.
[48] Janine van der Giessen, Dana Binyamin, Anna Belogolovski, Sigal Frishman, Kinneret Tenenbaum-Gavish, Eran Hadar, Yoram Louzoun, Maikel Petrus Peppelenbosch, Christien Janneke van der Woude, Omry Koren, et al. Modulation of cytokine patterns and microbiome during pregnancy in IBD. Gut, 69(3):473-486, 2020.
[49] Kei E Fujimura, Alexandra R. Sitarik, Suzanne Havstad, Din L. Lin, Sophia Levan, Douglas Fadrosh, Ariane R Panzer, Brandon LaMere, Elze Rackaityte, Nicholas W Lukacs, et al. Neonatal gut microbiota associates with childhood multisensitized atopy and T cell differentiation. Nature Medicine, 22(10):1187-1191, 2016.
[50] Ben P. Willing, Johan Dicksved, Jonas Halfvarson, Anders F. Andersson, Marianna Lucio, Zongli Zheng, Gunnar J'arnerot, Curt Tysk, Janet K Jansson, and Lars Engstrand. A pyrosequencing study in twins shows that gastrointestinal microbial profiles vary with inflammatory bowel disease phenotypes. Gastroenterology, 139(6):1844-1854, 2010.
[51] Xochitl C. Morgan, Timothy L. Tickle, Harry Sokol, Dirk Gevers, Kathryn L. Devaney, Doyle V. Ward, Joshua A. Reyes, Samir A. Shah, Neal LeLeiko, Scott B. Snapper, et al. Dysfunction of the intestinal microbiome in inflammatory bowel disease and treatment. Genome Biology, 13(9):1-18, 2012.
[52] Claire Duvallet, Sean Gibbons, Thomas Gurry, Rafael Irizarry, and Eric Alm. MicrobiomeHD: the human gut microbiome in health and disease. type: dataset, 2017.
[53] Derek Doran, Sarah Schulz, and Tarek R. Besold. What does explainable ai really mean? a new conceptualization of perspectives. arXiv preprint arXiv:1710.00794, 2017.
[54] Aditya Chattopadhay, Anirban Sarkar, Prantik Howlader, and Vineeth N. Balasubramanian. Grad-cam++: Generalized gradient-based visual explanations for deep convolutional networks. In 2018 IEEE Winter Conference on Applications of Computer Vision (WACV), pages 839-847. IEEE, 2018.
[55] Indrani Mukhopadhya, Richard Hansen, Emad M. El-Omar, and Georgina L. Hold. IBD-what role do Proteobacteria play? Nature Reviews Gastroenterology & Hepatology, 9(4):219-230, 2012.
[56] Youlian Zhou, Yan 758 He, Le Liu, Wanyan Zhou, PuWang, Han Hu, Yuqiang Nie, and Ye Chen. Alterations in gut microbial communities across anatomical locations in inflammatory bowel diseases. Frontiers in Nutrition, 8:58, 2021.
[57] Zhen He, JinjieWu, Junli Gong, Jia Ke, Tao Ding, Wenjing Zhao, WaiMing Cheng, Zhanhao Luo, Qilang He, Wanyi Zeng, et al. Microbiota in mesenteric adipose tissue from crohn's disease promote colitis in mice. Microbiome, 9(1):1-14, 2021.
[58] Amy O'callaghan and Douwe Van Sinderen. Bifidobacteria and their role as members of the human gut microbiota. Frontiers in Microbiology, 7:925, 2016.
[59] C. Picard, Jean Fioramonti, Arnaud Francois, Tobin Robinson, Francoise Neant, and C. Matuchansky. Bifidobacteria as probiotic agents-physiological effects and clinical benefits. Alimentary Pharmacology & Therapeutics, 22(6):495-512, 2005.
[60] Ting Zhang, Qianqian Li, Lei Cheng, Heena Buch, and Faming Zhang. Akkermansia muciniphila is a promising probiotic. Microbial Biotechnology, 12(6):1109-1125, 2019.
[61] Divya Sharma and Wei Xu. phyLoSTM: a novel deep learning model on disease prediction from longitudinal microbiome data. Bioinformatics, 2021.
[62] Laura Goodfellow, Marijn C. Verwijs, Angharad Care, Andrew Sharp, Jelena Ivandic, Borna Poljak, Devender Roberts, Christina Bronowski, A Christina Gill, Alistair C Darby, et al. Vaginal bacterial load in the second trimester is associated with early preterm birth recurrence: a nested case-control study. BJOG: An International Journal of Obstetrics & Gynaecology, 2021.
[63] Carole Roug'e, Oliver Goldenberg, Laurent Ferraris, Bernard Berger, Florence Rochat, Arnaud Legrand, Ulf B. Go'bel, Michel Vodovar, Marcel Voyer, Jean-Christophe Roz'e, et al. Investigation of the intestinal microbiota in preterm infants using different methods. Anaerobe, 16(4):362-370, 2010.
[64] Katri Korpela, Elin W Blakstad, Sissel J. Moltu, Kenneth Strpmmen, Britt Nakstad, Arild E. Rønnestad, Kristin Brokke, Per O. Iversen, Christian A. Drevon, and Willem de Vos. Intestinal microbiota development and gestational age in preterm neonates. Scientific Reports, 8(1):1-9, 2018.
[65] Francesca Turroni, Christian Milani, Sabrina Duranti, Gabriele Andrea Lugli, Sergio Bernasconi, Abelardo Margolles, Francesco Di Pierro, Douwe Van Sinderen, and Marco Ventura. The infant gut microbiome as a microbial organ influencing host well-being. Italian Journal of Pediatrics, 46(1):1-13, 2020.
[66] Oshrit Shtossel, Sondra Turjeman, Alona Riumin, Michael R Goldberg, Arnon Elizur, Hadar Mor, Omry Koren, and Yoram Louzoun. Recipient independent high accuracy FMT prediction and optimization in mice and humans. 2022.
[67] Yishay Pinto, Sigal Frishman, Sondra Turjeman, Adi Eshel, Meital Nuriel-Ohayon, Oshrit Shrossel, Oren Ziv, William Walters, Julie Parsonnet, Catherine Ley, et al. Gestational diabetes is driven by microbiota-induced inflammation months before diagnosis. Gut, 2023. William Falcon et al. Pytorch lightning. GitHub. Note: https://github(dot)com/PyTorchLightning/pytorch-lightning, 3, 2019.
[69] F. Pedregosa, G. Varoquaux, A. Gramfort, V. Michel, B. Thirion, O. Grisel, M. Blondel, P. Prettenhofer, R. Weiss, V. Dubourg, J. Vanderplas, A. Passos, D. Cournapeau, M. Brucher, M. Perrot, and E. Duchesnay. Scikit-learn: Machine learning in Python. Journal of Machine Learning Research, 12:2825-2830, 2011.
[70] Steve Lawrence, C. Lee Giles, Ah Chung Tsoi, and Andrew D. Back. Face recognition: A convolutional neural-network approach. IEEE Transactions on Neural Networks, 8(1):98-113, 1997.
[71] Pajau Vangay, Benjamin M Hillmann, and Dan Knights. Microbiome learning repo ([ml repo]): A public repository of microbiome regression and classification tasks. Gigascience, 8(5):giz042, 2019.
[72] Nan Qin, Fengling Yang, Ang Li, Edi Prifti, Yanfei Chen, Li Shao, Jing Guo, Emmanuelle Le Chatelier, Jian Yao, Lingjiao Wu, et al. Alterations of the human gut microbiome in liver cirrhosis. Nature, 513(7516):59-64, 2014.
[73] Human Microbiome Project Consortium et al. Structure, function and diversity of the healthy human microbiome. Nature, 486(7402):207, 2012.
[74] Jacques Ravel, Pawel Gajer, Zaid Abdo, G. Maria Schneider, Sara S. K. Koenig, Stacey L. McCulle, Shara Karlebach, Reshma Gorle, Jennifer Russell, Carol O. Tacket, et al. Vaginal microbiome of reproductive-age women. Proceedings of the National Academy of Sciences, 108(Supplement 1):4680-4687, 2011.
[75] Tommi Vatanen, Aleksandar D. Kostic, Eva d'Hennezel, Heli Siljander, Eric A. Franzosa, Moran Yassour, Raivo Kolde, Hera Vlamakis, Timothy D. Arthur, Anu-Maaria Ha‘ma’la-inen, et al. Variation in microbiome LPS immunogenicity contributes to autoimmunity in humans. Cell, 165(4):842-853, 2016.
[76] Daniel B. DiGiulio, Benjamin J. Callahan, Paul J. McMurdie, Elizabeth K. Costello, Deirdre J. Lyell, Anna Robaczewska, Christine L. Sun, Daniela S. A. Goltsman, Ronald J. Wong, Gary Shaw, et al. Temporal and spatial variation of the human microbiota during pregnancy. Proceedings of the National Academy of Sciences, 112(35):11060-11065, 2015.
[77] Tadayoshi Fushiki. Estimation of prediction error by using k-fold cross-validation. Statistics and Computing, 21(2):137-146, 2011.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant trees, data structures, and ML models will be developed and the scope of the terms tree, data structure, and ML model are intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A computer implemented method of determining a state of a medical condition of a subject, comprising:
computing a tree that includes representations computed from genetic sequences of a plurality of microbes within a microbiome sample of a subject, wherein the tree includes the representations of the plurality of microbes arranged according to a taxonomy hierarchy;
mapping the tree to a data structure that defines the taxonomy hierarchy and defines positions between neighboring representations of the plurality of microbes;
feeding the data structure into a machine learning model that processes neighboring representations according to position; and
obtaining the state of the medical condition of the subject as an outcome of the machine learning model.

2. The computer implemented method of claim 1, further comprising ordering elements of the data structure each denoting a type of microbe according to at least one similarity feature between different types of microbes at a same taxonomy level of the taxonomy hierarchy while preserving the structure of the tree.

3. The computer implemented method of claim 2, wherein the ordering is done per taxonomic category level, for positioning the representations of microbes that are more similar closer together and representation of microbes that are less similar are positioned further apart while preserving the structure of the tree.

4. The computer implemented method of claim 2, wherein the ordering is computed recursively per taxonomic category level.

5. The computer implemented method of claim 2, wherein the ordering is done for positioning taxa with similar frequencies relatively closer together and taxa with less similar frequencies further apart.

6. The computer implemented method of claim 2, wherein the at least one similarity feature includes similarity of frequency of microbes.

7. The computer implemented method of claim 6, wherein the at least one similarity feature is computed based on Euclidean distances.

8. The computer implemented method of claim 7, wherein the at least one similarity feature is computed by building a dendrogram based on the Euclidean distances computed for a hierarchical clustering of the representations of the microbes according to frequency.

9. The computer implemented method of claim 1, wherein the machine learning model is trained by:
obtaining a plurality of sample microbiome samples from a plurality of subjects;
computing the tree that includes representations computed from genetic sequences of the plurality of sample microbiome samples;
mapping the tree to the data structure;
creating a training dataset of a plurality of records, wherein a record comprises the data structure and a ground truth indication of the state of the medical condition of a subject from the plurality of subjects; and
training the machine learning model on the training dataset.

10. The computer implemented method of claim 1, wherein the tree is created by including each observed taxa of microbes of the microbiome sample in a leaf at a respective taxonomic level of the taxonomy hierarchy, and adding to each leaf a log-normalized frequency of the observed taxa of microbes, and each internal node includes an average of direct descendants of the internal node located at lower levels.

11. The computer implemented method of claim 1, wherein the data structure is implemented as a graph that includes values of the tree and an adjacent matrix, that are fed into a layer of a graph convolutional neural network implementation of the machine learning model, wherein an identity matrix is added with a learned coefficient, and output of the layer is fed into a fully connected layer that generates the outcome.

12. The computer implemented method of claim 1, wherein the data structure is represented as an image created by projecting the tree to a two dimensional matrix with a plurality of rows matching a number of different taxonomy category levels of the taxonomy hierarchy represented by the tree, and a column for each leaf, wherein the machine learning model is implemented as a convolutional neural network.

13. The computer implemented method of claim 12, wherein at each respective level of the different taxonomy category levels, a value of the two dimensional matrix at the respective level is set to the value of leaves below the level or a value at a higher level is set to have an average of values of a level below, and below a leaf values are set to zero, and wherein a level below includes a plurality of different values, a plurality of positions of the two dimensional matrix of the current level are set to an average of the plurality of different values of the level below.

14. The computer implemented method of claim 1, further comprising applying an explainable AI platform to the machine learning model for obtaining an estimate of portions of the data structure used by the machine learning model for obtaining the outcome, and projecting the portions of the data structure to the tree for obtaining an indication of which microorganisms of the microbiome most contributed to the outcome of the machine learning model.

15. The computer implemented method of claim 14, further comprising iterating the applying the explainable AI platform for each of a plurality of data structures of sequentially obtained microbiome samples, for creating a plurality of heatmaps each at a different color channel, and combining the plurality of heatmaps into a single multi-channel image, and projecting the multi-channel image on the tree.

16. The computer implemented method of claim 1, wherein a plurality of trees are computed for a plurality of microbiome samples obtained at spaced apart time intervals, wherein the plurality of trees are mapped to a plurality of image representations of the data structures corresponding to the spaced apart time intervals, the plurality of images are combined into a 3D image depicting temporal data, and the 3D image is fed into a 3D-CNN implementation of the machine learning model.

17. The computer implemented method of claim 1, further comprising pre-processing the genetic sequences by clustering the genetic sequences to create Amplicon Sequence Variants (ASVs), and creating a vector of the ASVs, wherein each entry of the vector represents a microbe at a certain taxonomy level of the taxonomy hierarchy and comprises the representation, wherein the tree is created from the vector by placing respective values of the vector at corresponding taxonomic category levels of the taxonomy hierarchy of the tree.

18. The computer implemented method of claim 17, further comprising computing a log-normalization of the ASV frequencies of the vector to obtain a log-normalized vector of ASV frequencies, wherein the tree includes the values of the log-normalized vector.

19. The computer implemented method of claim 18, further comprising recursively ordering the log-normalized ASV frequencies according to at least one similarity feature computed based on Euclidean distances between log-normalized ASV frequencies.

20. The computer implemented method of claim 1, wherein the medical state of the subject is an indication of a complication that arises during pregnancy that is linked to lifelong health risks, including at least one of: gestational diabetes, preeclampsia, preterm birth, and postpartum depression.

21. A computer implemented method of training a machine learning model for determining a state of a medical condition of a subject, comprising:
obtaining a plurality of sample microbiome samples from a plurality of subjects;
computing a tree that includes representations computed from genetic sequences of a plurality of microbes within the plurality of sample microbiome sample, wherein the tree includes the representations of the plurality of microbes arranged according to a taxonomy hierarchy;
mapping the tree to a data structure that defines the taxonomy hierarchy and defines positions between neighboring representations of the plurality of microbes;
creating a training dataset of a plurality of records, wherein a record comprises the data structure and a ground truth indication of the state of a medical condition of a subject from the plurality of subjects; and
training the machine learning model on the training dataset, wherein the machine learning model processes neighboring representations according to position, wherein the machine learning model generates the state of the medical condition of the subject in response to an input of a data structure computed from a microbiome sample obtained from the subject.

22. A system for determining a state of a medical condition of a subject, comprising:
computing a tree that includes representations computed from genetic sequences of a plurality of microbes within a microbiome sample of a subject, wherein the tree includes the representations of the plurality of microbes arranged according to a taxonomy hierarchy;
mapping the tree to a data structure that defines the taxonomy hierarchy and defines positions between neighboring representations of the plurality of microbes;
feeding the data structure into a machine learning model that processes neighboring representations according to position; and
obtaining the state of the medical condition of the subject as an outcome of the machine learning model,
wherein the machine learning model is trained by:
obtaining a plurality of sample microbiome samples from a plurality of subjects;
computing the tree that includes representations computed from genetic sequences of the plurality of sample microbiome samples;
mapping the tree to the data structure;
creating a training dataset of a plurality of records, wherein a record comprises the data structure a and a ground truth indication of the state of a medical condition of a subject from the plurality of subjects; and
training the machine learning model on the training dataset.
